(19) United States
(12) Patent Application Publication
(10) Pub. No.: US 2015/0299336 A1
(43) Pub. Date: Oct. 22, 2015

(54) THERAPEUTIC AND DIAGNOSTIC TARGET GENE IN ACUTE MYELOID LEUKEMIA

(71) Applicant: Ulrich G. Steidl, New Rochelle, NY (US)

(72) Inventor: Ulrich G. Steidl, New Rochelle, NY (US)

(73) Assignee: Albert Einstein College of Medicine of Yeshiva University, Bronx, NY (US)

(21) Appl. No.: 14/669,469

(22) Filed: Mar. 26, 2015

Related U.S. Application Data
(63) Continuation of application No. 14/113,405, filed on Dec. 16, 2013, now abandoned.
(60) Provisional application No. 61/481,924, filed on May 3, 2011.

(51) Int. Cl. C07K 16/40; C12N 15/113

(52) U.S. Cl. CPC C07K 16/40; C12N 15/1137; C12N 2310/14; C12N 2310/531

(57) ABSTRACT

Methods are provided for treating a cancer in a subject comprising administering to the subject an agent which inhibits expression of an HLX gene in the subject, or an agent which inhibits activity of an expression product of the HLX gene, and also for diagnosing a subject as likely to develop a cancer comprising determining whether a stem cell obtained from the subject expresses a HLX gene at a level in excess of predetermined control level. Kits therefor are also provided.

### CROSS-REFERENCE TO RELATED APPLICATIONS

This application claims benefit of U.S. Provisional Application No. 61/481,924, filed May 3, 2011, the contents of which are hereby incorporated by reference.

### STATEMENT OF GOVERNMENT SUPPORT

This invention was made with government support under grant number K99/R00CA131503 awarded by the National Cancer Institute. The government has certain rights in the invention.

### BACKGROUND OF THE INVENTION

Throughout this application various publications are referred to. Full citations for these references may be found at the end of the specification. The disclosures of these publications, and of all patents, patent application publications and books referred to herein, are hereby incorporated by reference in their entirety into the subject application to more fully describe the art to which the subject invention pertains.

Transcription factors are critical for the regulation of normal hematopoiesis as well as leukemogenesis. Several members of the Hox (Class I homeobox genes) family of transcription factors, which contain a conserved homeobox domain and are organized into 4 major gene clusters in humans, have been implicated in the functioning of hematopoietic stem and progenitor cells as well as for leukemic transformation and the generation of leukemia-initiating cells. Much less is known about the role of non-clustered (class II) homeobox genes in hematopoiesis and leukemia. The transcriptional analysis of purified stem and progenitor populations has recently been utilized as a powerful tool to identify critical regulators of stem and progenitor cell function and transformation to leukemia-initiating cells.

Analyzing hematopoietic stem and progenitor cells (HSPC) in a murine model of acute myeloid leukemia (AML), this laboratory found the non-clustered H2.0-like homeobox (Hlx) gene to be 4-fold upregulated compared to wildtype HSPC (Steidl, 2006).

Hlx is the highly conserved human/murine homologue of the homeobox gene H2.0, which was found to show tissue-specific expression throughout development in Drosophila melanogaster. Additional studies two decades ago detected Hlx expression in hematopoietic progenitors and in leukemic blasts of patients with AML, and a study of Hlx-deficient fetal liver cells suggested a decrease of colony-formation capacity. However, the function, if any, of Hlx in hematopoietic stem and progenitor cells, and its role, if any, in leukemia have not been studied.

The present invention addresses the need for novel anti-leukemia treatments and novel myelodysplastic syndrome treatments by providing, inter alia, treatments based on inhibition of HLX expression or of HLX expression products.

### SUMMARY OF THE INVENTION

A method of treating a cancer in a subject comprising administering to the subject an agent which inhibits expression of an HLX gene in the subject, or an agent which inhibits activity of an expression product of the HLX gene, so as to thereby treat the cancer.

Also provided is a method of diagnosing a subject as likely to develop a cancer comprising determining whether a stem cell obtained from the subject expresses a HLX gene at a level in excess of a predetermined control level, wherein HLX gene expressed in the stem cell in excess of the predetermined control level indicates that the subject is likely to develop the cancer.

Also provided is a method of diagnosing a subject as susceptible to developing a cancer comprising determining whether a stem cell obtained from the subject expresses a HLX gene at a level in excess of a predetermined control level, wherein HLX gene expressed in the stem cell in excess of the predetermined control level indicates that the subject is susceptible to developing the cancer.

Also provided is a method of diagnosing a subject as in need of aggressive anti-cancer therapy comprising determining whether a stem cell obtained from the subject expresses a HLX gene at a level in excess of a predetermined control level, wherein the HLX gene expressed in the stem cell in excess of the predetermined control level indicates that the subject is in need of aggressive anti-cancer therapy.

Also provided is a kit comprising written instructions and reagents for determining HLX gene expression levels in a biological sample obtained from a subject for determining the subject's susceptibility to acute myeloid leukemia or for determining if a subject is in need of aggressive anti-acute myeloid leukemia therapy.

A method is also provided of diagnosing a subject as likely to develop a cancer, or as susceptible to developing a cancer, comprising determining whether a sample obtained from the subject expresses a HLX gene at a level in excess of a predetermined control level, wherein HLX gene expressed in the sample determined to be in excess of the predetermined control level indicates that the subject is likely to develop the cancer or is susceptible to developing the cancer.

A method is also provided of diagnosing a subject as susceptible to developing a cancer, or as in need of aggressive anti-cancer therapy, comprising determining whether a sample obtained from the subject expresses one or more of the following genes at a level in excess of a predetermined control level for each gene (i) HLX, PGD, RASGRP4, ITGAM, PAK1, CD53, GCH1, GADD45B, NCOR2, SFXN3, PDLIM2, AIF1, PARVG, ZAK and IBRDC1, and/or expresses one or more of the following genes at a level below a predetermined control level for each gene (ii) ZNF451, AIG1, and GALC, wherein a determination of one or more of the genes in (i) expressed in the sample in excess of the predetermined control level indicates that the subject is susceptible to developing the cancer and wherein a determination of one or more of the genes in (ii) expressed in the sample below the predetermined control level indicates that the subject is susceptible to developing the cancer.

Also provided is a method of diagnosing a subject as suitable for an aggressive anti-cancer therapy comprising determining whether a sample obtained from the subject expresses a HLX gene at a level in excess of a predetermined control level, wherein the HLX gene expressed in the sample in excess of the predetermined control level indicates that the subject is suitable for an aggressive anti-cancer therapy, wherein the HLX gene expressed in the sample not in excess of the predetermined control level does not indicate that the subject is suitable for an aggressive anti-cancer therapy.

Also provided is a microarray comprising a plurality of nucleic acid probes, or a plurality of microarrays comprising a plurality of probes, with at least one of the nucleic acid probes of plurality of probes being specific for each of HLX, ZNF451, AIG1, GALC, PGD, RASGRP4, ITGAM, PAK1, CD53, GCH1, GADD45B, NCOR2, SFXN3, PDLIM2, AIF1, PARVG, ZAK and IBRDC1.

Also provided is a method of treating a cancer in a subject comprising administering to the subject an agent which inhibits expression of a PAK1 gene or of a BTG1 gene, or an agent which inhibits activity of an expression product of a PAK1 gene or of a BTG1 gene, so as to thereby treat the cancer.

Also provided is a method of diagnosing a subject as having a high-risk myelodysplastic syndrome comprising determining whether a sample obtained from the subject expresses a HLX gene at a level in excess of a predetermined control level, wherein HLX gene expressed in the sample in excess of the predetermined control level indicates that the subject has a high-risk myelodysplastic syndrome.

Also provided is a method of treating a myelodysplastic syndrome in a subject comprising administering to the subject an agent which inhibits expression of an HLX gene, or an agent which inhibits activity of an expression product of an HLX gene, so as to thereby treat the myelodysplastic syndrome.

A kit is provided comprising written instructions and reagents for determining HLX gene expression levels in a biological sample obtained from a subject for determining the subject's susceptibility to acute myeloid leukemia or high-risk myelodysplastic syndrome, or for determining if a subject is in need of aggressive anti-acute myeloid leukemia therapy.

A kit is provided comprising written instructions and reagents for determining HLX gene expression levels and PAK1 gene expression levels in a biological sample obtained from a subject for determining the subject's susceptibility to acute myeloid leukemia or high-risk myelodysplastic syndrome, or for determining if a subject is in need of aggressive anti-acute myeloid leukemia therapy.

Also provided is a kit comprising written instructions and reagents for determining expression levels of the genes HLX, ZNF451, AIG1, GALC, PGD, RASGRP4, ITGAM, PAK1, CD53, GCH1, GADD45B, NCOR2, SFXN3, PDLIM2, AIF1, PARVG, ZAK and IBRDC1 in a biological sample obtained from a subject for determining the subject's susceptibility to acute myeloid leukemia or high-risk myelodysplastic syndrome, or for determining if a subject is in need of aggressive anti-acute myeloid leukemia therapy.

### BRIEF DESCRIPTION OF THE DRAWINGS

FIG. 1A-1E. Hlx overexpression impairs hematopoietic reconstitution, eliminates functional long-term hematopoietic stem cells, and leads to persistence of Lin−CD34−kit− cells. (1A) Schematics of lentiviral vectors (control and Hlx-IRES-GFP). (1B) Increased protein expression of Hlx in Lin−kit+ cells after transduction with Hlx-expressing lentivirus and sorting of GFP+ cells. (1C,1D) Control- or Hlx-IRES-GFP-transduced Lin−Kit+ cells (Ly5.2) together with spleen cells from congenic wild-type mice (Ly5.1) were transplanted into lethally irradiated congenic wild-type recipients (Ly5.1) (N=7). Data from 12 weeks after transplantation are shown. Representative FACS plots and individual data points of total GFP+ cells in peripheral blood (1C), and Lin−kit+Sca+Flk2-Thy1lo LT-HSC in bone marrow (1D) are shown. The mean contribution of GFP+ to total donor cells is indicated by horizontal lines in the panels on the right. (1E) Analysis of GFP+ cells in total bone marrow cells from recipients transplanted with Hlx-transduced Lin−Kit+ cells after 12 weeks. The gating strategy and relative percentages of lin-negative as well as CD34−kit-cells are indicated.

FIG. 2A-2F. Hlx overexpression confers serial replating capacity to Lin−CD34−kit− cells. (2A) Primary colony formation assay (left panel) and serial replating assay (right panel) of Lin−kit+Sca1+ cells after transduction with control lentivirus or Hlx lentivirus. GFP-positive colonies derived from control cells (white bars) and Hlx-overexpressing cells (black bars) are shown. Error bars indicate one standard deviation. Statistical significance is indicated (* means p<0.05, and ** means p<0.005, N=3). (2B) Photograph of entire tissue culture dishes after 5th plating shows enlarged size of colonies derived from the Hlx-transduced cells. Scale bar indicates 1 cm. (2C) FACS analysis demonstrates that Hlx overexpression leads to a decrease of phenotypically immature CD34+kit+ cells, and increases the CD34−kit− population. A representative FACS plot is shown. (2D) The frequency of each population within total GFP-positive cells is shown (I=CD34+kit+, II=CD34+kit−, III=CD34−kit+, IV=CD34−kit−). Control cells (white bars) and Hlx-overexpressing cells (black bars). Error bars indicate one standard deviation. Statistical significance is indicated (* means p<0.05, N=3). (2E) Whole plate photographs of colonies derived from sorted cells from each population (I, II, III, IV) are shown. Scale bars indicate 1 cm. (2F) Serial replating assay of each sorted population (I, II, III, IV). Colony numbers after 2nd plating (white bars), 3rd plating (gray bars) and in 4th plating (black bars) are shown.

FIG. 3A-3E. Hlx induces a partial myelo-monocytic differentiation block. (3A) FACS analysis of GFP+CD34−kit− cells after the primary colony-forming assay. Cells were additionally stained with Gr-1, Mac1, F4/80, Ter119, B220, and CD3 antibodies as indicated in the figure. Relative percentages of cells in the indicated gates are given and show a significant reduction of mature myelomonocytic cells derived from the cells overexpressing Hlx. (3B) FACS analysis of cells derived from control-transduced or Hlx-transduced LSK cells after culture in methylcellulose with 25 ng/ml GM-CSF. Relative percentages of cells are given for each gate and show a lower number of cells expressing mature myelomonocytic markers. (3C) Representative morphology of cells from 3B, confirming a partial myelomonocytic differentiation block. Numerous cells with immature morphology can be found in the colonies derived from cells transduced with Hlx (indicated by arrows). Scale bar shows 100 um. (3D) FACS analysis of cells derived from control-transduced or Hlx-transduced LSK cells after culture in methylcellulose with 100 ng/ml M-CSF. Relative percentages of cells are given for each gate and show a lower number of cells expressing mature myelomonocytic markers. (3E) Representative morphology of cells from (3D), which show a monocytic differentiation block. Cells with immature morphology are indicated by arrows. Scale bar shows 100 μm.

FIG. 4A-4M. Hlx downregulation inhibits acute myeloid leukemia. Lentiviruses expressing short hairpins directed against Hlx (sh Hlx) or a control (sh control) were used to downregulate Hlx in URE cells. (4A) Western blotting shows a >80% reduction of Hlx protein in Hlx knockdown cells. (4B) Clonogenic assay of URE cells treated with sh control or sh Hlx cells. 1,000 cells each were seeded and cultured in M3434 methylcellulose medium for 10 days and GFP-positive colonies were counted. Error bars indicate S.D. (N=3). (4C) Cell proliferation kinetics were determined by MTS assays (N=5), and (4D) manual cell counts using trypan blue exclusion (N=3) in sh control cells (white bars) and sh Hlx cells (black bars). Error bars indicate S.D. (4E, 4F) Cell surface marker analysis after treatment with 100 ng/ml recombinant GM-CSF in suspension culture for 3 days. Relative percentages of cells in the indicated gates are given, and show a decrease of immature kit+ cells and an increase of kit-Gr1+ cells (4E), and an increase of Mac1+ cells (4F). (4G) Morphology of cells after treatment with 100 ng/ml recombinant GM-CSF for 3 days. Scale bar shows 100 μm. Cells with maturation signs are indicated by arrows. (4H) Analysis of the relative percentages of viable cells (DAPI-negative/AnexinV-negative), apoptotic cells (DAPI-negative/AnnexinV-positive) and necrotic cells (entire DAPI-positive) in sh control cells (white bars) and sh Hlx cells (black bars). Error bars indicate S.D. (N=3). P values are indicated. (4I) Cell cycle status of sh control and sh Hlx leukemia cells measured by EdU assays. Percentages of cells in G0/G1 (white bars), S (gray bars), and G2/M (black bars) phase of cell cycle are displayed. Statistical significance is indicated. (4J) Transplantation of URE cells transduced with sh control or sh Hlx into NSG mice. 1 million (left panel, N=10 in sh control and N=9 in sh Hlx) or 5 million cells (right panel, N=9 in sh control and N=8 in sh Hlx) were retroorbitally injected into NSG mice after sublethal irradiation (250 cGy). Kaplan-Meier curves of overall survival of recipient mice (sh control: solid line; sh Hlx: dashed line) are displayed and show a clear survival advantage for mice who received cells with Hlx inhibition. P values (log-rank) are indicated. (4K) Hierarchical clustering of genes differentially expressed in URE leukemia cells upon Hlx knockdown. Only genes with −log 10(p) value <0.05 and a mean difference >0.5 were considered differentially expressed. After filtering out unannotated and duplicate genes, genes were clustered by hierarchical, Euclidean distance, complete linkage clustering. Expression levels are color-coded (log(2) scale as indicated above the cluster tree) with lighter gray indicating low, and black indicating high expression. (4L) Enrichment map representation of cellular processes perturbed in leukemia cells upon Hlx knockdown. Enriched gene sets are represented as nodes (black circles) connected by edges (dark gray links) denoting the degree of gene set overlap. The node size is proportional to the number of genes in the gene set and the edge thickness represents the number of genes that overlap between gene sets. The color intensity of the nodes indicates the statistical significance of enrichment of a particular gene set. Groups of functionally related gene sets are circled in grey and labeled. (4M) Select genes altered by Hlx inhibition in URE leukemia cells. Up-regulated genes are shown in black and down-regulated genes are shown in gray. Their involvement in regulation of cell cycle/proliferation, cell death, and myeloid differentiation is indicated. Upward arrows indicate an increase, downward arrows a decrease in the listed process.

FIG. 5A-5F. HLX is overexpressed in patients with acute myeloid leukemia and correlates with poor overall survival. (5A) Waterfall plot of relative expression (log 2) of Hlx of 344 patients with acute myeloid leukemia (“AML”) in comparison to CD34-enriched bone marrow cells from 12 healthy donors. (5B) Box plot summary of the HLX expression data (log(2) scale) shown in 5A. HLX expression is significantly higher in patients with AML in comparison to CD34+ cells from healthy donors (p=1.9×10⁻⁹). The median expression values (bold lines), 25th and 75th percentile (bottom and top of box), and the minimum and maximum (lower and upper whiskers) of both groups are shown. (5C) Kaplan-Meier survival plots comparing overall survival (OS) of patients with high versus low HLX expression in a combined dataset of 601 patients with AML (GSE10358, GSE12417 (U133plus2.0) and GSE14468). Patients with high expression of HLX show drastically inferior clinical outcome. The p value (log-rank test) is indicated. (5D-5F) Kaplan-Meier survival plots comparing overall survival (OS) of patients with high versus low HLX expression in molecularly defined subsets of AML. Curves for HLX low (black) and HLX high (different colors) are shown. High expression of HLX is associated with significantly inferior clinical outcome in all subsets. P values (log-rank test) are given. (5D) AML patients with no detectable mutations of the FLT3 gene. (5E) AML patients with mutant NPM1. (5F) AML patients with mutant CEBPA.

FIG. 6. Schematics of transplantation assays. Lin−Kit+ cells from wild-type C57BL/6J (Ly5.2) mice were sorted (upper panel) and transduced with lentivirus (control or Hlx) in the presence of IL-3, IL-6, and SCF. 24 hours after transduction, 5×10⁴ lentivirus-transduced Lin−Kit+ cells (Ly5.2) together with 2.5×10⁵ spleen cells from congenic wild-type mice (Ly5.1) were transplanted into lethally irradiated congenic wild-type recipients (C57BL/6J:Pep3b, Ly5.1) (middle left). 40 hours after transduction, the frequency of GFP-positive cells was analyzed by flow-cytometry. Transduction efficiency was near 50% in both lentivirus transductions (middle right). Peripheral blood was analyzed 8 weeks and 12 weeks after transplantation, and recipient mice were sacrificed and their bone marrow was analyzed 12 weeks after transplantation by flow-cytometry as shown (lower panels). Results are shown in FIG. 1 and FIG. 7.

FIG. 7. Flow cytometric analysis 12 weeks after transplantation. Representative FACS plots and individual data points of total GFP-positive cells within total short-term HSC (ST-HSC; Thy1loFLk2+LSK), multipotent progenitors (MPP; Thy1-FLk2+LSK), common myeloid progenitors (CMP; Lin−kit+Sca−1−FcγR1oCD341o), granulocyte/monocyte progenitors (GMP; Lin−kit+Sca−1−FcγRhiCD341o) and megakaryocyte/erythrocyte progenitors (MEP; Lin−kit+Sca−1−FcγR−CD34−) as defined in FIG. 6 are shown.

FIG. 8. Homing is not affected by Hlx overexpression. 8×10⁴ lentivirus-transduced Lin−Kit+ cells from wild-type C57BL/6 mice (Ly5.2) were transplanted into lethally irradiated congenic wild-type recipients (Ly5.1). Bone marrow mononuclear cells from recipients were stained with PE conjugated Ly5.1 (recipient) antibody and APC conjugated Ly5.2 (donor) antibody and analyzed by flow-cytometry 24 hour after transplantation. A representative FACS plot is shown in the left panel. The frequency of GFP-positive cells among the donor population (Ly5.1−Ly5.2+), was assessed. Data summary is shown in the right panel. Error bars indicate S.D. (N=3).

FIG. 9. Apoptosis is not induced by Hlx overexpression. Sorted LSK cells from wild-type FVB/nJ mice were transduced with control lentivirus or Hlx lentivirus. 5 days after transduction, cells were stained by PE conjugated Annexin V (BD Pharmingen) and DAPI, and analyzed by flow cytometry. The frequency of Annexin V positive cells and/or DAPI positive cells is indicated.

FIG. 10. Photographs of representative colonies derived from control lentivirus-transduced and Hlx-lentivirus-transduced LSK cells. Scale bars indicate 200 μm.

FIG. 11. 1×10⁶ Hlx-overexpressing GFP+CD34−kit− cells from the 5th plating were transplanted into NSG mice after sublethal (250 cGy) irradiation. Representative FACS plots of peripheral blood 7 weeks after transplantation with clearly detectable GFP+ cells are shown.

FIG. 12. GFP+kit−CD34− cells and Lineage (Gr-1, Ter119, F4/80, CD19, B220, CD3)-negative GFP+kit−CD34− cells from the first plate were sorted and seeded into M3434 methylcellulose media. GFP-positive colonies were scored after 10 days and show drastically increased clonogenicity of Hlx lentivirus-transduced cells.

FIG. 13. Validation of differential mRNA expression of candidate genes upon Hlx knockdown. For each indicated gene, expression level in sh Hlx cells was compared to sh control cells. Fold changes are shown according to microarray (white bar) and real-time PCR (black bar) data. Downward-pointing bars indicate decreased expression and upward-pointing bars indicate increased expression in sh Hlx cells. Primers are described in Table 1.

FIG. 14. Complete enrichment map of cellular processes perturbed in leukemia cells upon Hlx knockdown (p<0.05 and FDR<0.25). Enriched gene sets are represented as nodes (black circles) connected by edges (dark gray links) denoting the degree of gene set overlap. The node size is proportional to the number of genes in the gene set and the edge thickness represents the number of genes that overlap between gene sets. The color intensity of the nodes indicates the statistical significance of enrichment of a particular gene set. Groups of functionally related gene sets are circled in grey and labeled.

FIG. 15A-15F. Kaplan-Meier plots comparing overall survival (OS) of patients with high versus low HLX expression in different published datasets of patients with AML. Patients with high expression of HLX show inferior clinical outcome in each individual dataset. (15A) GSE12417(U133A). (15B) GSE12417(U133plus2.0). (15C) GSE10358. (15D) GSE14468. P values (log-rank test) are indicated. (15E) Kaplan-Meier plot of overall survival (irrespective of HLX status) in datasets GSE12417 (U133plus2.0), GSE14468 and GSE10358. The graph shows superimposable survival curves (p=0.4636, log-rank test), indicating very similar overall survival in each of these three datasets. (15F) Kaplan-Meier plot of overall survival (irrespective of HLX status) of the combined patients from datasets GSE12417(U133plus2.0) and GSE14468 and GSE10358, in comparison to patients from dataset GSE12417(U133A). The plot shows that patients from the GSE12417(U133A) cohort had overall a significantly poorer clinical outcome (p=0.0009) than patients from all other cohorts.

FIG. 16. Kaplan-Meier plots comparing overall survival of AML patients with low versus high overall score of the Hlx core signature (“Hlx core signature LOW” (black line) and “Hlx core signature HIGH” (gray line)). Patients with an Hlx core signature score above the median (“Hlx core signature HIGH”) have a significantly inferior overall survival (p<0.0001).

FIG. 17. Replating data showing “immortalization” of myeloid progenitors and unlimited clonogenicity with Hlx expression.

FIG. 18. Inhibitory effect of HLX in several human AML cell lines.

FIG. 19A-19B. 19A: This figure shows data indicating that Btg1 and Pak1 are functionally critical downstream genes of Hlx and, (19B) mediate the anti-leukemic effect of Hlx inhibition. As such, Btg1 and Pak1 are therapeutic targets.

FIG. 20A-20B. 20A: HLX regulates an entire signature of gene expression (17 genes), and this is signature is strongly prognostic in terms of overall survival (as is Hlx expression itself). 20B: The lower panel shows the signature being tested and validated in an additional independent patient cohorts.

FIG. 21. PAK1 expression levels are of prognostic relevance in AML, but only in combination with high HLX levels, indicating functional cooperativity.

FIG. 22. PAK1 levels are correlated with HLX expression in AML patients, and overexpression of Hlx leads to increased Pak1 levels in myeloid stem and progenitor cells.

FIG. 23. HLX is specifically elevated in patients with high-risk myelodysplastic syndromes (MDS) in a subset of patients classified as RAEB-2 (refractory anemia with excess of blasts 2). This subgroup has the most aggressive type of disease and is most likely to progress to overt AML. HLX elevation can be used to identify patients who are most likely to progress to AML and thus require treatment and can be a therapeutic target in MDS patients in general, too.

### DETAILED DESCRIPTION OF THE INVENTION

A method of treating a cancer in a subject comprising administering to the subject an agent which inhibits expression of an HLX gene in the subject, or an agent which inhibits activity of an expression product of the HLX gene, so as to thereby treat the cancer.

Also provided is a method of diagnosing a subject as likely to develop a cancer comprising determining whether a stem cell obtained from the subject expresses a HLX gene at a level in excess of a predetermined control level, wherein HLX gene expressed in the stem cell in excess of the predetermined control level indicates that the subject is likely to develop the cancer.

Also provided is a method of diagnosing a subject as susceptible to developing a cancer comprising determining whether a stem cell obtained from the subject expresses a HLX gene at a level in excess of a predetermined control level, wherein HLX gene expressed in the stem cell in excess of the predetermined control level indicates that the subject is susceptible to developing the cancer.

Also provided is a method of diagnosing a subject as in need of aggressive anti-cancer therapy comprising determining whether a stem cell obtained from the subject expresses a HLX gene at a level in excess of a predetermined control level, wherein the HLX gene expressed in the stem cell in excess of the predetermined control level indicates that the subject is in need of aggressive anti-cancer therapy.

In an embodiment of the methods, the cancer is acute myeloid leukemia. In an embodiment of the methods, the cancer is acute myeloid leukemia and the anti-cancer therapy is an anti-acute myeloid leukemia therapy.

In an embodiment of the methods, the subject has been diagnosed as being of intermediate cytogenetic risk for AML.

In an embodiment of the methods, the subject has a NPM1 mutation or a CEBPA mutation, or the subject does not have a FLT3 mutation.

In an embodiment of the methods, determining the level of expression of HLX gene is effected by quantifying HLX gene RNA transcript levels. In an embodiment the transcript is an mRNA.

In an embodiment of the methods, RNA transcript levels are quantified using quantitative reverse transcriptase PCR.

In an embodiment of the methods, the method comprises administering to the subject the agent which inhibits expression of HLX gene. In an embodiment of the methods, the method comprises administering to the subject the agent which inhibits activity of an expression product of the HLX gene.

In an embodiment of the methods, the expression product of the HLX gene is H2.0-like homeobox protein.

In an embodiment of the methods, the agent is an siRNA or an shRNA directed to the HLX gene.

In an embodiment of the methods, the HLX gene comprises consecutive nucleotide residues having the sequence set forth in SEQ ID NO:1.

Also provided is a kit comprising written instructions and reagents for determining HLX gene expression levels in a biological sample obtained from a subject for determining the subject's susceptibility to acute myeloid leukemia or for determining if a subject is in need of aggressive anti-acute myeloid leukemia therapy.

A method is provided for treating a cancer in a subject comprising administering to the subject an agent which inhibits expression of an HLX gene, or an agent which inhibits activity of an expression product of an HLX gene, so as to thereby treat the cancer.

In an embodiment, the method comprises administering to the subject the agent which inhibits expression of HLX gene. In an embodiment, the method comprises administering to the subject the agent which inhibits activity of an expression product of the HLX gene.

A method is also provided of diagnosing a subject as likely to develop a cancer, or as susceptible to developing a cancer, comprising determining whether a sample obtained from the subject expresses a HLX gene at a level in excess of a predetermined control level, wherein HLX gene expressed in the sample determined to be in excess of the predetermined control level indicates that the subject is likely to develop the cancer or is susceptible to developing the cancer.

A method is also provided of diagnosing a subject as susceptible to developing a cancer, or as in need of aggressive anti-cancer therapy, comprising determining whether a sample obtained from the subject expresses one or more of the following genes at a level in excess of a predetermined control level for each gene (i) HLX, PGD, RASGRP4, ITGAM, PAK1, CD53, GCH1, GADD45B, NCOR2, SFXN3, PDLIM2, AIF1, PARVG, ZAK and IBRDC1, and/or expresses one or more of the following genes at a level below a predetermined control level for each gene (ii) ZNF451, AIG1, and GALC,

wherein a determination of one or more of the genes in (i) expressed in the sample in excess of the predetermined control level indicates that the subject is susceptible to developing the cancer and wherein a determination of one or more of the genes in (ii) expressed in the sample below the predetermined control level indicates that the subject is susceptible to developing the cancer.

In an embodiment, a determination of no genes in (i) expressed in the sample in excess of the predetermined control level and no genes in (ii) expressed in the sample below the predetermined control level, does not indicate the subject is susceptible to developing the cancer or as in need of aggressive anti-cancer therapy.

In an embodiment, a determination of at least 2, 3, 4, 5, 6, 7, 8, 9, 10, 11, 12, 13, or 14 genes, or wherein 15 genes, in (i) expressed in the sample in excess of the predetermined control level and/or wherein a determination of at least 2 genes or wherein 3 genes in (ii) expressed in the sample below the predetermined control level, indicates the subject is susceptible to developing the cancer or as in need of aggressive anti-cancer therapy.

A method is provided of diagnosing a subject as suitable for an aggressive anti-cancer therapy comprising determining whether a sample obtained from the subject expresses a HLX gene at a level in excess of a predetermined control level, wherein the HLX gene expressed in the sample in excess of the predetermined control level indicates that the subject is suitable for an aggressive anti-cancer therapy, wherein the HLX gene expressed in the sample not in excess of the predetermined control level does not indicate that the subject is suitable for an aggressive anti-cancer therapy.

In an embodiment of the methods, the cancer is an acute myeloid leukemia. In an embodiment, the aggressive anti-cancer therapy is an anti-acute myeloid leukemia therapy. In an embodiment, the subject has been diagnosed as being of intermediate cytogenetic risk for AML. In an embodiment, the subject has a NPM1 mutation or a CEBPA mutation, or wherein the subject does not have a FLT3 mutation.

In an embodiment of the methods, determining the level of expression of the HLX gene, or other gene, is effected by quantifying gene RNA transcript levels. In an embodiment, the gene RNA transcript is mRNA. In an embodiment the gene RNA transcript levels are quantified by quantifying the corresponding nucleic acid(s), such as cDNA. In an embodiment, RNA transcript levels are quantified using quantitative reverse transcriptase PCR.

In an embodiment of the methods, the expression product of the HLX gene is a human H2.0-like homeobox protein. In an embodiment of the methods, the HLX gene comprises consecutive nucleotide residues having the sequence set forth in SEQ ID NO:1.

In an embodiment of the methods, the agent is an siRNA or an shRNA directed to the HLX gene.

In an embodiment of the methods, the sample comprises a blood sample, a bone marrow sample, or a stem cell.

In an embodiment of the methods, the method comprises determining whether the sample obtained from the subject expresses all of the following genes is expressed at a level in excess of a predetermined control level for each gene: HLX, PGD, RASGRP4, ITGAM, PAK1, CD53, GCH1, GADD45B, NCOR2, SFXN3, PDLIM2, AIF1, PARVG, ZAK and IBRDC1, and determining whether the sample obtained from the subject expresses all of the following genes is expressed at a level below a predetermined control level for each gene: ZNF451, AIG1, GALC.

In an embodiment of the methods, the methods further comprise using a microarray to determine the expression level of HLX gene or the expression level of the genes selected from HLX, ZNF451, AIG1, GALC, PGD, RASGRP4, ITGAM, PAK1, CD53, GCH1, GADD45B, NCOR2, SFXN3, PDLIM2, AIF1, PARVG, ZAK and IBRDC1.

Also provided is a microarray comprising a plurality of nucleic acid probes, or a plurality of microarrays comprising a plurality of probes, with at least one of the nucleic acid probes of plurality of probes being specific for each of HLX, ZNF451, AIG1, GALC, PGD, RASGRP4, ITGAM, PAK1, CD53, GCH1, GADD45B, NCOR2, SFXN3, PDLIM2, AIF1, PARVG, ZAK and IBRDC1.

Also provided is a method of treating a cancer in a subject comprising administering to the subject an agent which inhibits expression of a PAK1 gene or of a BTG1 gene, or an agent which inhibits activity of an expression product of a PAK1 gene or of a BTG1 gene, so as to thereby treat the cancer.

Also provided is a method of diagnosing a subject as having a high-risk myelodysplastic syndrome comprising determining whether a sample obtained from the subject expresses a HLX gene at a level in excess of a predetermined control level, wherein HLX gene expressed in the sample in excess of the predetermined control level indicates that the subject has a high-risk myelodysplastic syndrome. In an embodiment of the methods, the high-risk myelodysplastic syndrome is refractory anemia with excess of blasts II (RAEB II).

Also provided is a method of treating a myelodysplastic syndrome in a subject comprising administering to the subject an agent which inhibits expression of an HLX gene, or an agent which inhibits activity of an expression product of an HLX gene, so as to thereby treat the myelodysplastic syndrome. In an embodiment of the methods, the myelodysplastic syndrome is refractory anemia with excess of blasts II (RAEB II). In an embodiment of the methods, the myelodysplastic syndrome is RA, RARS, or RAEB-1.

In an embodiment of the methods, the agent is a small organic molecule of less than 2000 daltons, an antibody directed against PAK1 or BTG1 or a fragment of said antibody, or a nucleic acid molecule that effects RNAi and is directed to the PAK1 gene or BTG1 gene. In an embodiment of the methods, the agent is an siRNA or an shRNA directed to the PAK1 gene or BTG1 gene.

A kit is provided comprising written instructions and reagents for determining HLX gene expression levels in a biological sample obtained from a subject for determining the subject's susceptibility to acute myeloid leukemia or high-risk myelodysplastic syndrome, or for determining if a subject is in need of aggressive anti-acute myeloid leukemia therapy.

A kit is provided comprising written instructions and reagents for determining HLX gene expression levels and PAK1 gene expression levels in a biological sample obtained from a subject for determining the subject's susceptibility to acute myeloid leukemia or high-risk myelodysplastic syndrome, or for determining if a subject is in need of aggressive anti-acute myeloid leukemia therapy.

In an embodiment, the kits comprise a microarray having (i) a nucleic acid probe thereon specific for a transcript of an HLX gene or (ii) a nucleic acid probe thereon specific for a transcript of an HLX gene and a nucleic acid probe thereon specific for a transcript of an PAK1 gene transcript.

In an embodiment, the kits comprise a set of forward and reverse PCR primers specific for a region of the HLX gene comprising a portion encoding a transcript of the HLX gene for which the nucleic acid probe is specific.

In an embodiment, the kits comprise a set of forward and reverse PCR primers specific for a region of the PAK1 gene comprising a portion encoding a transcript of the PAK1 gene for which the nucleic acid probe is specific.

A kit is provided comprising written instructions and reagents for determining expression levels of the genes HLX, PGD, RASGRP4, ITGAM, PAK1, CD53, GCH1, GADD45B, NCOR2, SFXN3, PDLIM2, AIF1, PARVG, ZAK, IBRDC1, ZNF451, AIG1, and GALC,

in a biological sample obtained from a subject for determining the subject's susceptibility to acute myeloid leukemia or high-risk myelodysplastic syndrome, or for determining if a subject is in need of aggressive anti-acute myeloid leukemia therapy.

In an embodiment, the kits comprise a plurality of sets of forward and reverse PCR primers, each set specific for a region of one of the recited genes comprising a portion encoding a transcript of the gene for which the nucleic acid probe is specific.

An aggressive anti-cancer therapy is determined by those of skill in the art, such as physicians, based on the cancer, and means that a less-aggressive anti-cancer therapy is available. For example, aggressive anti-cancer therapy in AML could comprise a stem-cell transplantation. For example, an aggressive anti-cancer therapy in could comprise an aggressive chemotherapy.

As used herein, HLX gene is a human gene encoding H2.0-like homeobox protein. (Convention has upper case “HLX” as the human gene and “Hlx” as non-human equivalents).

In an embodiment, the HLX gene has RefSeq Accession no. NM_{—}021958.3.

| | |
|---|---|
| 1 | aaaactttgg gagtttttag agacgagttt tttttttttt ctattacttt tccccccccc |
| 61 | taactaacgg actattattg ttgttgtttt aaatttagct cttagggctt agctatttgg |
| 121 | gttttcttgc ggtgtccggc tcccgtctcc ctggctcccc cgcccgccct gcggccccag |
| 181 | cgcccctcgc tctcatccag cccgcgagga gtgcgggcgc cgcgccgcct ttaaagcgag |
| 241 | gccagggagc gaggcggtga ccggccgaga tccggccctc gcctcctccc tcggtggcgc |
| 301 | tagggctccc ggcctctctt cctcagtgcg ggcggagaag cgaaagcgga tcgtcctcgg |
| 361 | ctgccgccgc cttctccggg actcgcgcgc ccctccccgc gcgcccaccc acccagtccg |
| 421 | gctggactgc ggcagccgcg cggctcaccc cggcaggatg ttcgcagccg ggctggctcc |
| 481 | cttctacgcc tccaacttca gcctctggtc ggccgcttac tgctcctcgg ccggcccagg |
| 541 | cggctgctcc ttccccttgg accccgccgc cgtcaaaaag ccctccttct gcatcgcaga |
| 601 | cattctgcac gccggcgtgg gggatctggg ggcggccccg gagggcctgg caggggcctc |
| 661 | ggccgccgcc ctcaccgcgc acttgggctc ggttcacccg cacgcctctt tccaagcggc |
| 721 | ggccagatcc ccgcttcgac ccaccccagt ggtggcgccc tccgaagtcc cggctggctt |
| 781 | cccgcagcgg ctgtctccgc tctcagccgc ctaccaccac catcacccgc aacaacaaca |
| 841 | gcagcagcaa cagccgcagc agcaacagcc tccgcctccg ccccgggctg gcgccctgca |
| 901 | gcccccggcc tcggggacgc gagtggttcc gaacccccac cacagtggct ctgccccggc |
| 961 | cccctccagc aaagacctca aatttggaat tgaccgcatt ttatctgcag aatttgaccc |
| 1021 | aaaagtcaaa gaaggcaaca cgctgagaga tctcacttcc ctgctaaccg gtgggcggcc |
| 1081 | cgccggggtg cacctctcag gcctgcagcc ctcggccggc cagttcttcg catctctaga |
| 1141 | tcccattaac gaggcttctg caatcctgag tcccttaaac tcgaacccaa gaaattcagt |
| 1201 | tcagcatcag ttccaagaca cgtttccagg tccctatgct gtgctcacga aggacaccat |
| 1261 | gccgcagacg tacaaaagga agcgttcatg gtcgcgcgct gtgttctcca acctgcagag |
| 1321 | gaaaggcctg gagaaaaggt ttgagattca gaagtacgtg accaagccgg accgaaagca |
| 1381 | gctggcggcg atgctgggcc tcacggacgc acaggtgaag gtgtggttcc agaaccggcg |
| 1441 | gatgaagtgg cggcactcca aggaggccca ggcccaaaag gacaaggaca aggaggctgg |
| 1501 | cgagaagcca tcaggtggag ccccggctgc ggatggcgag caggacgaga ggagccccag |
| 1561 | ccgttctgaa ggcgaggctg agagcgagag cagcgactcc gagtccctgg acatggcccc |
| 1621 | cagcgacacg gagcggactg aggggagtga gcgttctctg caccaaacaa cagttattaa |
| 1681 | ggccccggtc actggcgccc tcattaccgc cagcagtgct gggagtggtg ggagcagcgg |
| 1741 | cggcggcggc aatagtttca gcttcagcag cgccagcagt cttagtagca gcagcaccag |
| 1801 | tgcgggttgc gccagcagcc ttggcggcgg cggcgcctcg gagcttctcc ctgcaacaca |
| 1861 | gcccacagcc agcagcgctc ccaaaagccc cgagccagcc caaggcgcgc ttggctgctt |
| 1921 | atagactgta ctagggcgga ggggatccgg gccttgcgtg cagcctccca accatgggct |
| 1981 | gggttttgtg cttactgtat gttggcgact tggtagggca ggagacgcag cgtggagcct |
| 2041 | acctcccgac attcacgctt cgccccacgc tgctccgact ggctgcagcg gacactgccc |
| 2101 | aaagcagagg ggagtctcag tgtcctgcta gccagccgaa cacttctctc cggaagcagg |
| 2161 | ctggttcgac tgtgaggtgt ttgactaaac tgtttctctg actcgcccca gaggtcgtgg |
| 2221 | ctcaaaggca cttaggacgc cttaaatttg taaataaaat gtttactacg gtttgtaaaa |
| 2281 | aaaaaaaaaa aaaaaaaaaa aaaaaaaa |

(NCBI Reference Sequence: NM_{—}021958.3; SEQ ID NO:1). In an embodiment, each t in the above sequence is replaced with a u.

As used herein, PAK1 is p21 protein (Cdc42/Rac)-activated kinase (a serine/threonine-protein kinase enzyme) that in humans is encoded by the PAK1 gene.

As used herein, BTG1 (B cell translocation gene) is a protein that in humans is encoded by the BTG1 gene.

In an embodiment, an siRNA (small interfering RNA) used as an agent in the methods or compositions described herein is directed to HLX and comprises a portion which is complementary to an mRNA sequence encoded by NCBI Reference Sequence: NM_{—}021958.3, and the siRNA is effective to inhibit expression of Homo sapiens H2.0-like homeobox (HLX). In an embodiment, the siRNA as used in the methods or compositions described herein in regard to inhibiting PAK1 (being directed to a sequence encoding PAK1) comprises a portion which is complementary to an mRNA sequence encoding Pak1. In an embodiment, the encoding sequence comprises NCBI Reference Sequence: NM_{—}001128620.1 or NCBI Reference Sequence: NM_{—}002576.4, and the siRNA is effective to inhibit expression of human PAK1. In an embodiment, the siRNA as used in the methods or compositions described herein in regard to inhibiting BTG1 (being directed to a sequence encoding BTG1) comprises a portion which is complementary to an mRNA sequence encoding BTG1. In an embodiment, the encoding sequence comprises NCBI Reference Sequence: NM_{—}001731.2, and the siRNA is effective to inhibit expression of human BTG1.

In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. In an embodiment, the overhang is UU. The siRNA can be 5′ phosphorylated or not and may be modified with any of the known modifications in the art to improve efficacy and/or resistance to nuclease degradation. In a non-limiting embodiment, the siRNA can be administered such that it is transfected into one or more cells.

In one embodiment, a siRNA of the invention comprises a double-stranded RNA comprising a first and second strand, wherein one strand of the RNA is 80, 85, 90, 95 or 100% complementary to a portion of an RNA transcript of a gene encoding Homo sapiens H2.0-like homeobox (HLX) (or of PAK1 or BTG1 as appropriate, mutatis mutandis). Thus, in an embodiment, the invention encompasses an siRNA comprising a 19, 20 or 21 nucleotide first RNA strand which is 80, 85, 90, 95 or 100% complementary to a 19, 20 or 21 nucleotide portion, respectively, of an RNA transcript of an HLX gene. In embodiment, the second RNA strand of the double-stranded RNA is also 19, 20 or 21 nucleotides, respectively, a 100% complementary to the first strand. In another embodiment, a siRNA of the invention comprises a double-stranded RNA wherein one strand of the RNA comprises a portion having a sequence the same as a portion of 18-25 consecutive nucleotides of an RNA transcript of a gene encoding Homo sapiens H2.0-like homeobox (HLX). In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification.

In one embodiment, RNAi inhibition of HLX is effected by an agent which is a short hairpin RNA (“shRNA”). The shRNA is introduced into the cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene, in the present case HLX. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In one embodiment, inhibition of HLX is effected by an agent which is an antibody or by a fragment of an antibody. As used herein, the term “antibody” refers to complete, intact antibodies, “fragment of an antibody” refers to Fab, Fab′, F(ab)₂, and other fragments thereof, or an ScFv, which bind the antigen of interest, in this case an HLX gene, or which bind Hlx. Complete, intact antibodies include, but are not limited to, monoclonal antibodies such as murine monoclonal antibodies, polyclonal antibodies, chimeric antibodies, human antibodies, and humanized antibodies.

Various forms of antibodies may be produced using standard recombinant DNA techniques (Winter and Milstein, Nature 349: 293-99, 1991). For example, “chimeric” antibodies may be constructed, in which the antigen binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567; Morrison et al., Proc. Natl. Acad. Sci. 81: 6851-55, 1984). Chimeric antibodies reduce the immunogenic responses elicited by animal antibodies when used in human clinical treatments. In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences into which the regions responsible for specific antigen-binding have been inserted (see, e.g., PCT patent application WO 94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in antibodies for use in human therapies, and are less likely to elicit unwanted immune responses. Primatized antibodies can be produced similarly.

Another embodiment of the antibodies employed in the compositions and methods of the invention is a human antibody directed against HLX, or a fragment of such antibody, which can be produced in nonhuman animals, such as transgenic animals harboring one or more human immunoglobulin transgenes. Such animals may be used as a source for splenocytes for producing hybridomas, for example as is described in U.S. Pat. No. 5,569,825.

Fragments of the antibodies described herein and univalent antibodies may also be used in the methods and compositions of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab)₂), whether any of the above are covalently or non-covalently aggregated, so long as the aggregation is capable of specifically reacting with a particular antigen or antigen family.

In an embodiment, the agents of the invention as described herein are administered in the form of a composition comprising the agent and a carrier. The term “carrier” is used in accordance with its art-understood meaning, to refer to a material that is included in a pharmaceutical composition but does not abrogate the biological activity of pharmaceutically active agent(s) that are also included within the composition. Typically, carriers have very low toxicity to the animal to which such compositions are to be administered. In some embodiments, carriers are inert.

In one embodiment of the methods, the HLX expression level or activity level of the gene product thereof (or of PAK1 or BTG1 protein, mutatis mutandis) is detected using a detectable agent. As used herein, a “detectable agent” is any agent that binds to HLX gene or to Hlx and which can be detected or observed, when bound, by methods known in the art. In non-limiting examples, the detectable agent can be an antibody or a fragment of an antibody, which is itself detectable, e.g. by a secondary antibody, or which is labeled with a detectable marker such as a radioisotope, a fluorophore, a dye etc. permitting detection of the presence of the bound agent by the appropriate machine, or optionally in the case of visually detectable agents, with the human eye. In an embodiment, the amount of detectable agent can be quantified.

As used herein, a “cancer” is a disease state characterized by the presence in a subject of cells demonstrating abnormal uncontrolled replication. In a preferred embodiment, the cancer is a leukemia. In a most preferred embodiment, the cancer is acute myeloid leukemia. As used herein, “treating” a cancer, or a grammatical equivalent thereof, means effecting a reduction of, amelioration of, or prevention of further development of one or more symptoms of the disease, or placing the cancer in a state of remission, or maintaining it in a state of remission.

As used herein a “leukemia” is an art-recognized cancer of the blood or bone marrow characterized by an abnormal increase of immature white blood cells called “blasts”. The specific condition of acute myeloid leukemia (AML) is a cancer of the myeloid line of blood cells, characterized by the rapid growth of abnormal white blood cells that accumulate in the bone marrow and interfere with the production of normal blood cells.

The myelodysplastic syndromes (MDS, formerly known as preleukemia) are a collection of hematological conditions that involve ineffective production (or dysplasia) of the myeloid class of blood cells. Patients with MDS often develop severe anemia and require frequent blood transfusions. In most cases, the disease worsens and the patient develops cytopenias (low blood counts) due to progressive bone marrow failure. In about one third of patients with MDS, the disease transforms into acute myelogenous leukemia (AML), usually within months to a few years. The myelodysplastic syndromes are all disorders of the stem cell in the bone marrow. RAEB II is indicated by the presence of 10-19% blasts, and has a poorer prognosis than RAEB I (5-9% blasts).

In an embodiment, the stem cell obtained from the subject is obtained by obtaining a sample from the subject. As used herein, a “sample” of a cancer or of a tumor is a portion of the cancer or of the tumor, respectively, for example as obtained by a biopsy. In the case of a leukemia, or AML, the preferred sample is bone marrow, or is derived from bone marrow, or is blood or is derived from blood. In an embodiment, the sample is, or comprises, a stem cell. As used herein a “sample derived from blood” or a “sample derived from bone marrow” is a sample which has been treated chemically and/or mechanically, but in such a manner not to alter HLX expression levels or activity levels which might be contained therein.

In an embodiment, the microarray comprises probes attached via surface engineering to a solid surface by a covalent bond to a chemical matrix (via, in non-limiting examples, epoxy-silane, amino-silane, lysine, polyacrylamide). Suitable solid surface can be, in non-limiting examples, glass or a silicon chip, a solid bead forms of, for example, polystyrene. As used herein, unless otherwise specified, a microarray includes both solid-phase microarrays and bead microarrays. In an embodiment, the microarray is a solid-phase microarray. In an embodiment, the microarray is a plurality of beads microarray. In an embodiment, the microarray is a spotted microarray. In an embodiment, the microarray is an oligonucleotide microarray. The nucleic acid probes (e.g. oligonucleotide probes) of the microarray may be of any convenient length necessary for unique discrimination (is specific for) of target gene transcripts. In non-limiting examples, the probes are 20 to 30 nucleotides in length, 31 to 40 nucleotides in length, 41 to 50 nucleotides in length, 51 to 60 nucleotides in length, 61 to 70 nucleotides in length, or 71 to 80 nucleotides in length. In an embodiment, the target sample (e.g. gene mRNA transcripts), or nucleic acids derived from the target sample, such as cDNA, are contacted with a detectable marker, such as one or more fluorophores, under conditions permitting the detectable marker to attach to the target sample or nucleic acids derived from the target sample. Such fluorophores are well known in the art, for example cyanine 3, cyanine 5. In an embodiment, the target hybridized to the probe can be detected by conductance, mass spectrometry (including MALDI-TOF), or electrophoresis. The microarray can be manufactured by any method known in the art including by photolithography, pipette, drop-touch, piezoelectric (ink-jet), and electric techniques.

If desired, mRNA in the sample can be enriched with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994), hereby incorporated by reference). In a non-limiting example, once bound, poly(A)+mRNA is eluted from the affinity column using 2 mM EDTA/0.1% SDS. Methods for preparing total and poly(A)+RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)), the contents of both of which are incorporated herein. RNA may be isolated from samples of eukaryotic cells by procedures that involve lysis of the cells and denaturation of the proteins contained therein. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979) hereby incorporated by reference). Poly(A)+RNA can be selected by selection with oligo-dT cellulose (see Sambrook et al, MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

As used herein “likely” in describing an occurrence means more likely than not. As used herein, “susceptible to” in describing a condition means more likely to develop the condition in a situation than a majority of the population from which the subject is drawn.

As used herein a “predetermined level” with regard to a quantity is the level of the quantity determined from one or more suitable control(s). In an embodiment the suitable control is a subject who does not have the relevant cancer and/or is not susceptible to the relevant cancer, or is a tissue or cell of such a subject. In an embodiment, the cancer that the subject does not have and/or is not susceptible to is acute myeloid leukemia.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

It is disclosed herein that HLX is overexpressed in the majority of patients with acute myeloid leukemia (“AML”) and is associated with poor clinical outcome. It is also disclosed herein that HLX increases clonogenicity and inhibits differentiation, and that the inhibition of HLX has an anti-leukemic effect. This study identifies Hlx as a novel class II homeobox gene which is critically involved in the pathogenesis of acute myeloid leukemia, and suggests that HLX is a prognostic and therapeutic target.

### Example 1

### Hlx Overexpression Impairs Hematopoietic Reconstitution, Eliminates Functional Long-Term Hematopoietic Stem Cells, and Leads to Persistence of a Small Progenitor Population

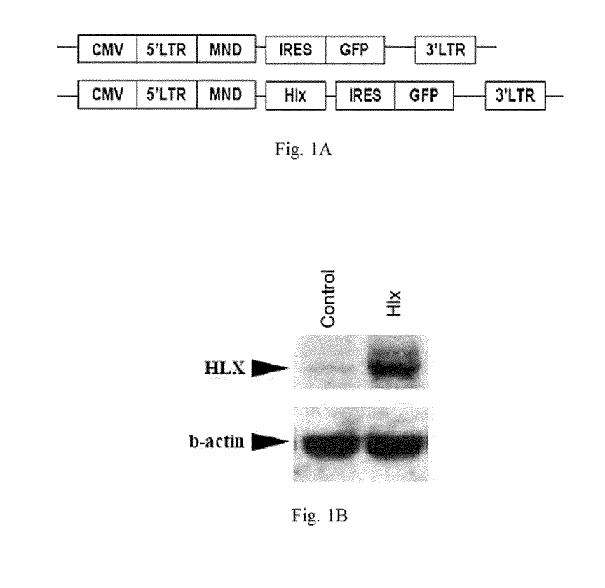

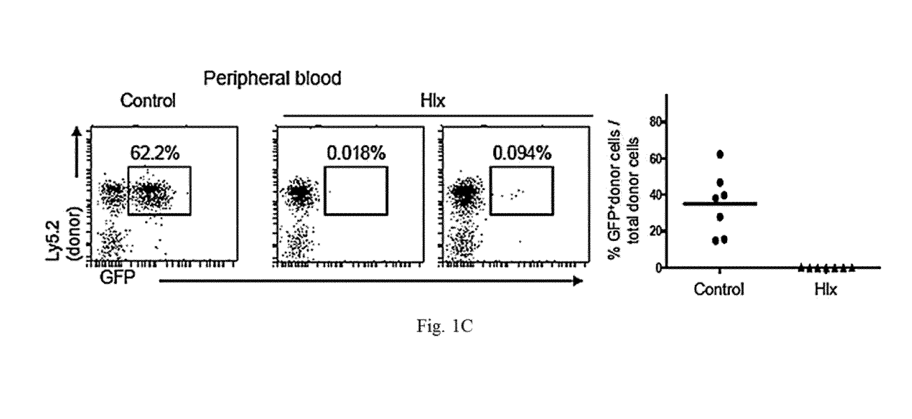

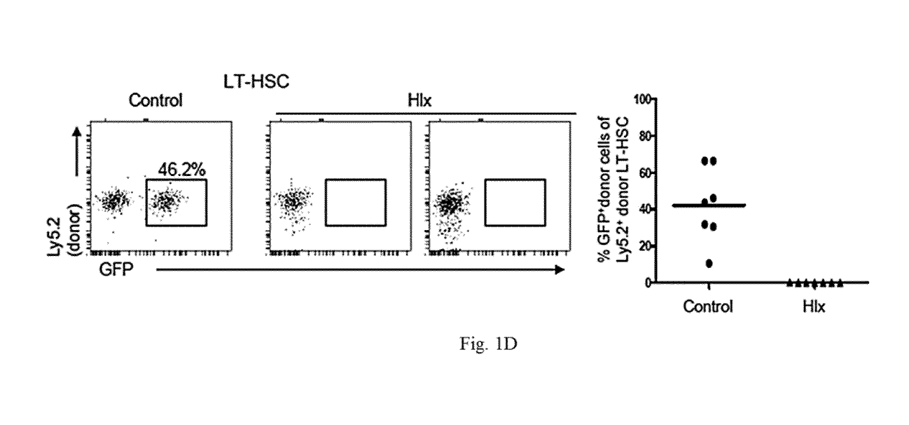

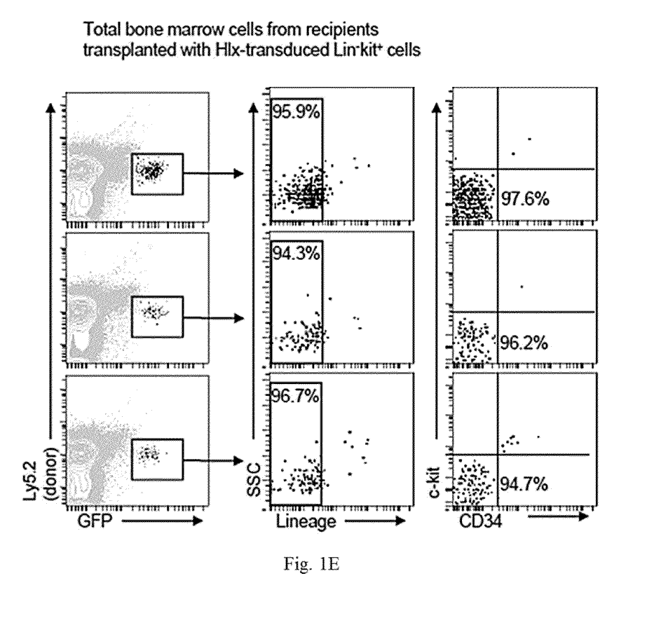

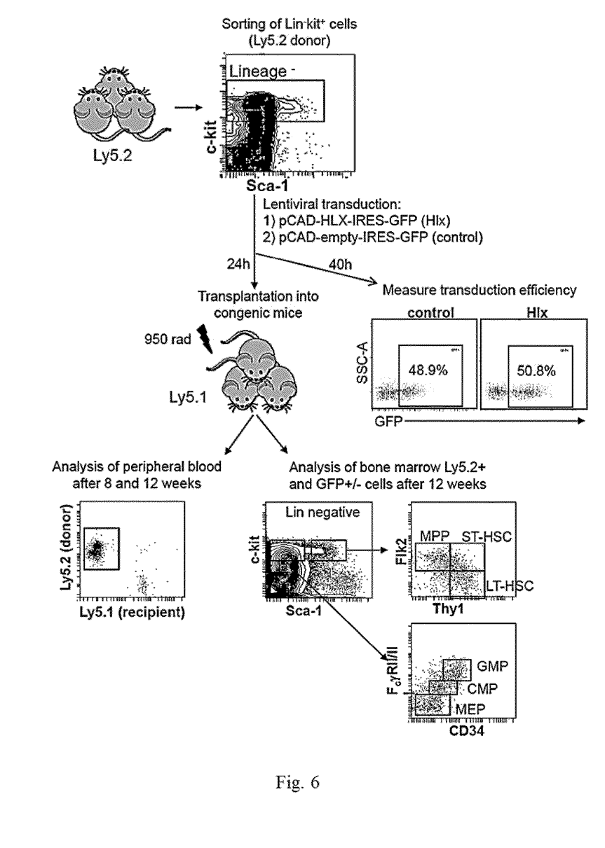

To examine the functional consequences of elevated Hlx levels on hematopoiesis, a lentiviral overexpression system was utilized (FIG. 1A+B). Lineage-negative (Lin−), c-Kit+ cells were sorted from the bone marrow of wildtype mice and transduced cells with either a lentivirus expressing GFP as a control or lentivirus expressing Hlx and GFP, and then transplanted cells into lethally irradiated congenic recipient mice. At the time of transplantation, transduction efficiency of control lentivirus and Hlx lentivirus was comparable, with both at approximately 50% (FIG. 6). Twenty-four hours post-transplantation, both control GFP-positive cells and Hlx-overexpressing GFP-positive Ly5.2 donor cells were detected in the bone marrow at similar frequencies (42.3% and 40.3%, respectively), indicating homing of the transplanted cells. Eight weeks and twelve weeks after transplantation, hematopoietic multilineage reconstitution was evaluated in the peripheral blood. Both groups engrafted robustly with an average donor chimerism of Ly5.2 cells of 80% (SD: 10%) and 85% (SD: 9%) in the control and Hlx group, respectively. However, while mice transplanted with control cells showed on average a percentage of 35% (SD: 17%) GFP-positive donor cells in the peripheral blood, mice transplanted with Hlx-transduced cells displayed drastically less GFP-positive donor cells (average: 0.07%, SD: 0.06%), demonstrating a severe defect of Hlx-overexpressing cells in hematopoietic reconstitution (FIG. 1C).

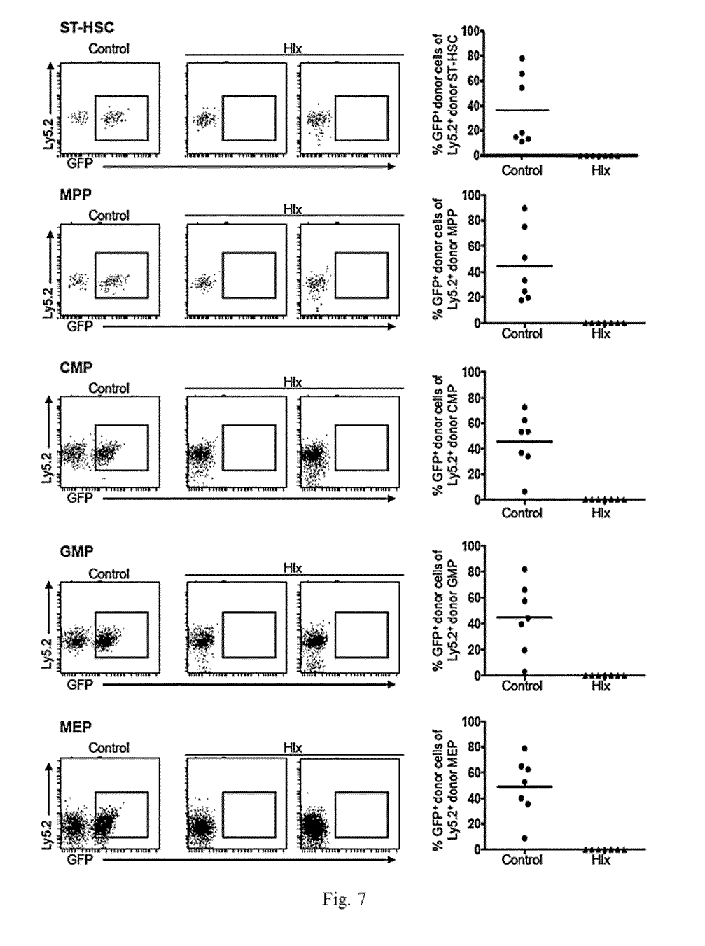

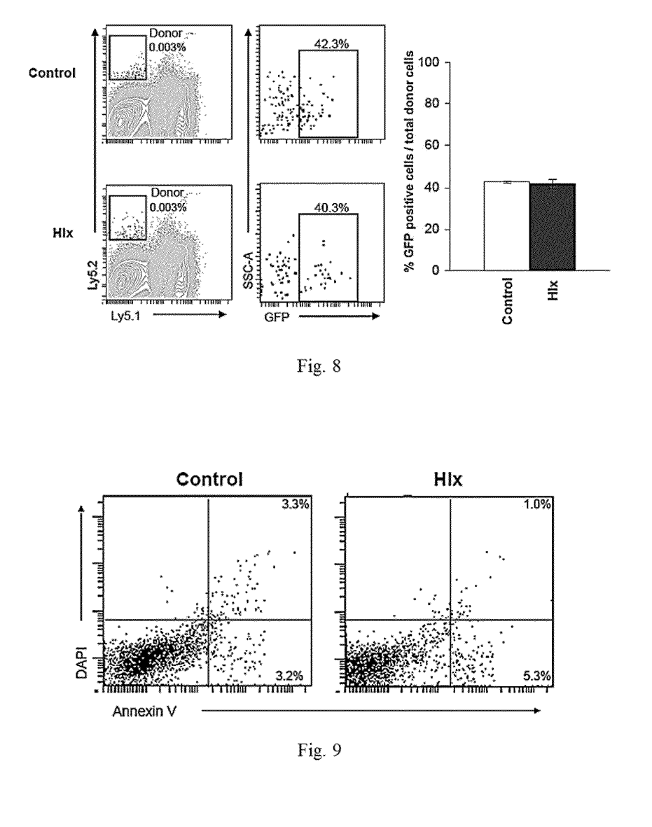

To determine the cellular compartments in which Hlx was effective stem and progenitor cells in the recipient bone marrow (BM) were analyzed. Strikingly, in the mice transplanted with Hlx-expressing cells, GFP-positive long-term HSC (LT-HSC; Thy1loFLk2-LSK (Lin−Sca1+Kit+)) could not be detected, while an average of 42% (SD:20%) GFP-positive LT-HSC was found in the control mice (FIG. 1D). Furthermore, in contrast to control animals, GFP-positive Hlx-expressing short-term HSC (ST-HSCs; Thy1loFLk2+LSK), multipotent progenitors (MPP; Thy1−FLk2+LSK), common myeloid progenitors (CMP; Lin−kit+Sca−1−FcγRloCD341o), granulocyte/monocyte progenitors (GMP; Lin−kit+Sca−1−FcγRhiCD341o) and megakaryocyte/erythrocyte progenitors (MEP; Lin−kit+Sca−1−FcγR−CD34−) were not found, indicating that Hlx acts at the level of the earliest hematopoietic stem cells. (FIG. 7). Given the lack of LT-HSC as well as more committed progenitors, HLX-GFP-positive transduced KL cells were analyzed by AnnexinV/DAPI staining to determine if Hlx overexpression might act by induction of apoptosis or necrosis in the transplanted KL cells. Both the control as well as Hlx-overexpressing cells displayed the same low percentage of apoptotic/necrotic cells (FIG. 9), indicating that Hlx acts by a mechanism other than induction of apoptosis or necrosis. Consequently, total bone marrow of recipient animals was we searched for alternative donor-derived GFP-positive cell populations persisting upon Hlx overexpression. Strikingly, a small population of GFP-positive, CD45.2(Ly.5.2)-positive cells was detected, which were still present 12 weeks after transplantation and were lineage-negative, CD34-negative, and c-Kit-negative (FIG. 1E). To characterize this cell population further, a series of experiments testing their cell biological properties, including clonogenic and differentiation capacities were performed.

Hlx Confers Increased Serial Clonogenicity to CD34−Kit− Hematopoietic Cells.

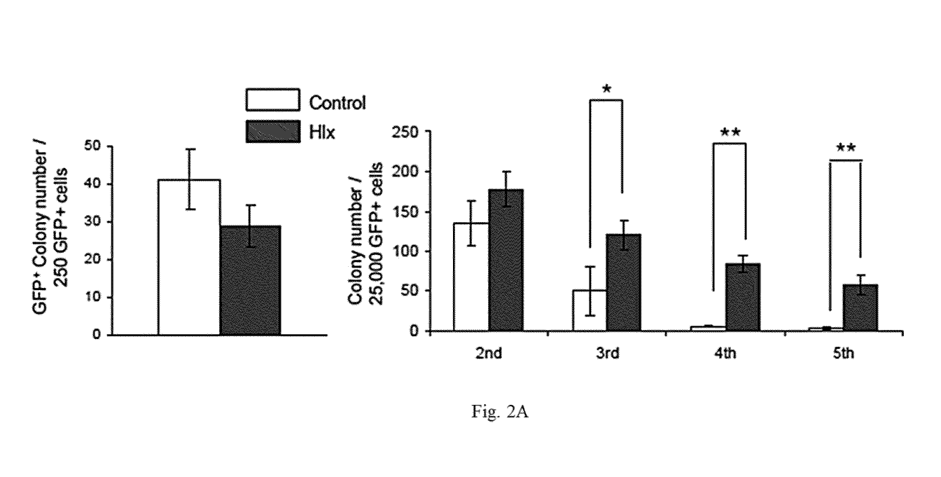

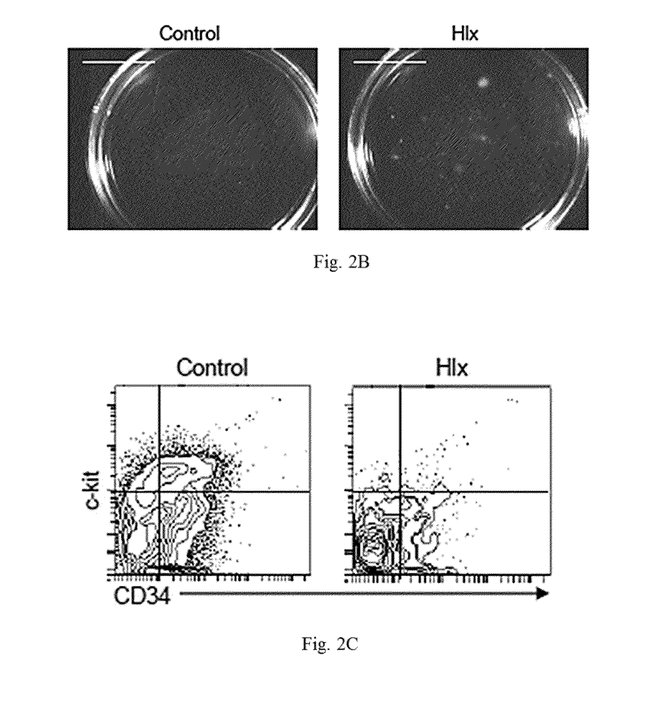

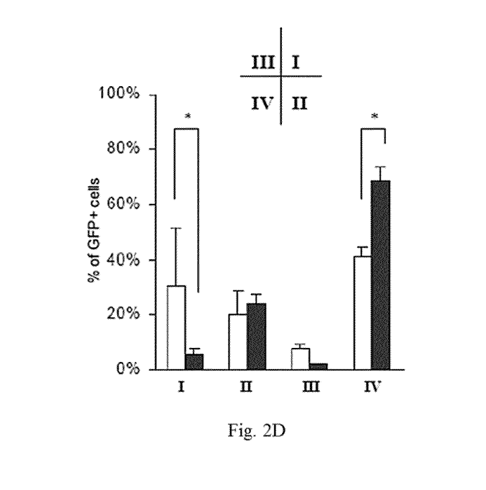

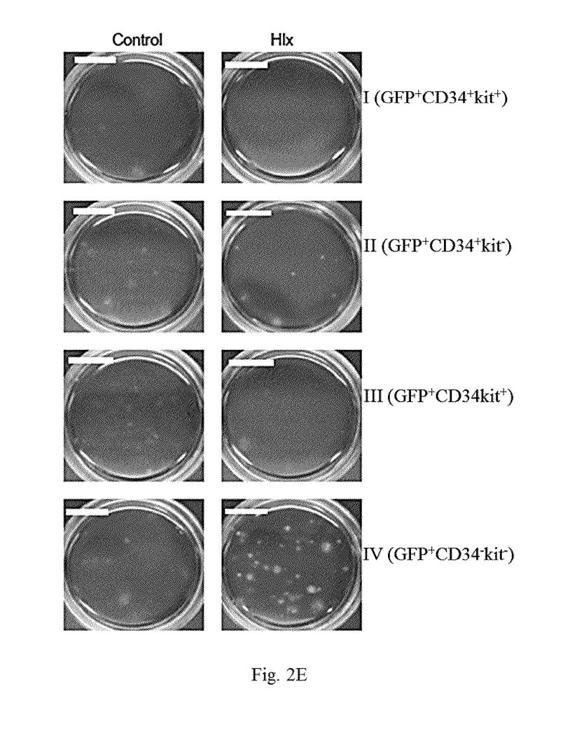

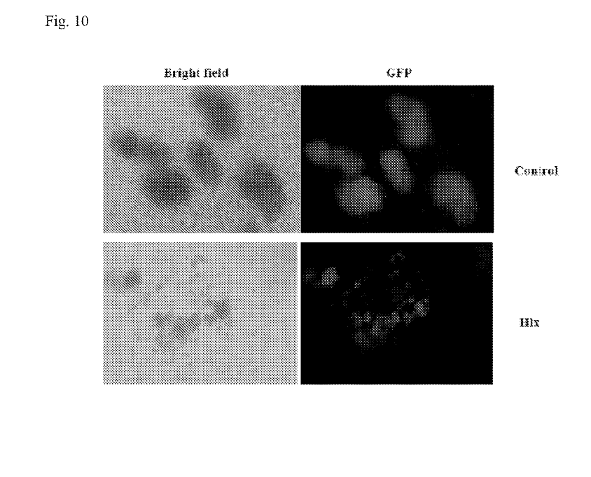

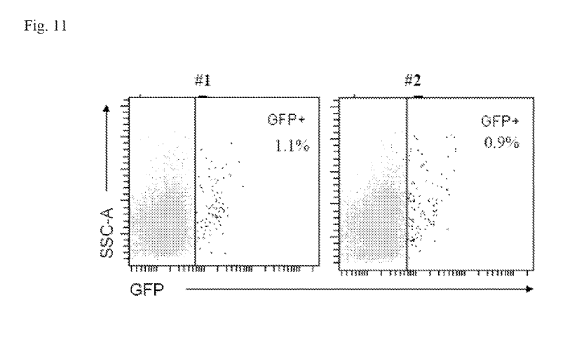

To test the effect of Hlx overexpression on hematopoietic stem and progenitor cells, in vitro colony formation assays of transduced LSK cells were performed. Hlx-transduced LSK cells formed slightly fewer colonies than control-transduced LSK cells (FIG. 2A). Colonies derived from Hlx-transduced LSK cells were also smaller (FIG. 10). To evaluate long-term clonogenicity of Hlx-overexpressing cells, serial-replating assays were performed. Strikingly, Hlx overexpressing cells showed greater clonogenic capacity in the 2nd and 3rd plating in comparison to control-transduced cells, and maintained serial clonogenicity in the 4th and 5th plating (FIG. 2A). Colonies were not only more numerous than control but noticeably larger in size after five platings (FIG. 2B). Cell surface marker expression of cells was analyzed from the initial plating and it was noticed that Hlx overexpression led to a decrease of c-kit+ cells, similar to the in vivo phenotype, and an increased proportion of phenotypically more mature CD34−Kit− cells in comparison to control-transduced cells (FIG. 2C+D). To determine which cellular subpopulation(s) conferred the increased clonogenic capacity, equal numbers of CD34+kit+ cells, CD34+kit− cells, CD34−kit+ cells, and CD34−kit− cells were sorted from the first plating, and subjected each individual population to colony formation assays. Only CD34−Kit− cells derived from Hlx-overexpressing cells formed a larger number of colonies in comparison to control cells, while all other populations did not display significant clonogenicity (FIG. 2E). Furthermore, the Hlx-overexpressing GFP+CD34−Kit− cells showed serial replating capacity through 4 rounds, while all other populations exhausted significantly earlier (FIG. 2F). Finally, when the serially-replating, Hlx-overexpressing GFP+CD34−kit− cells were injected after the fourth plating into irradiated NOD-SCID-IL2Rgamma null (NSG) mice, GFP-positive cells could still be detected after 7 weeks in the peripheral blood (FIG. 11). These data indicate that increased levels of Hlx confer long-term clonogenicity to a population of CD34−kit− cells.

Hlx Induces a Partial Myelo-Monocytic Differentiation Block

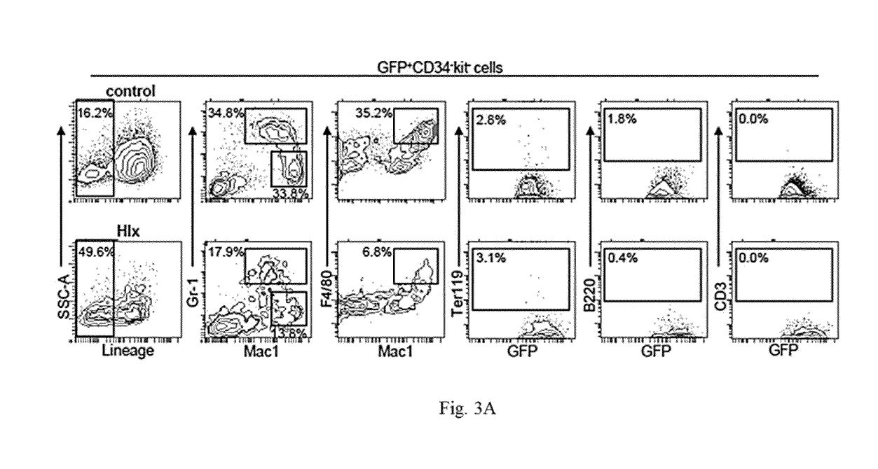

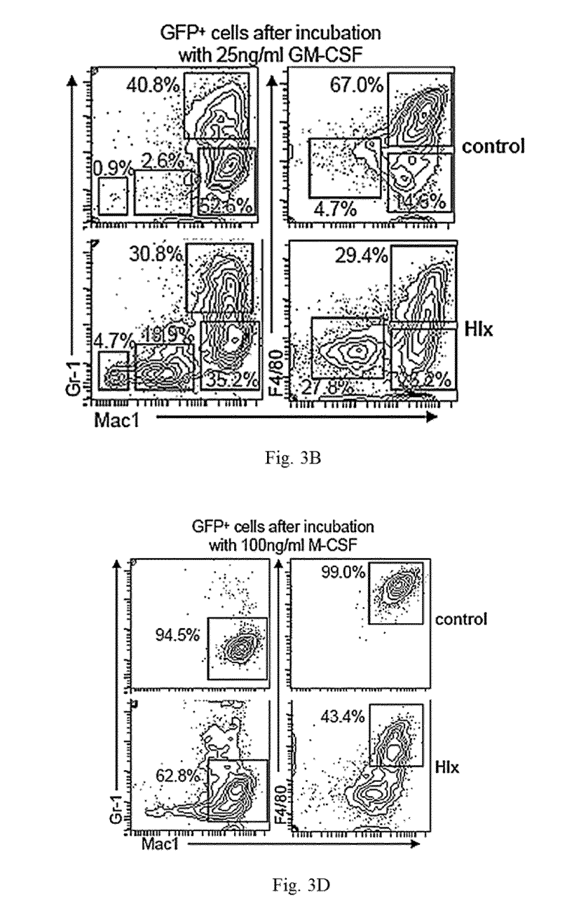

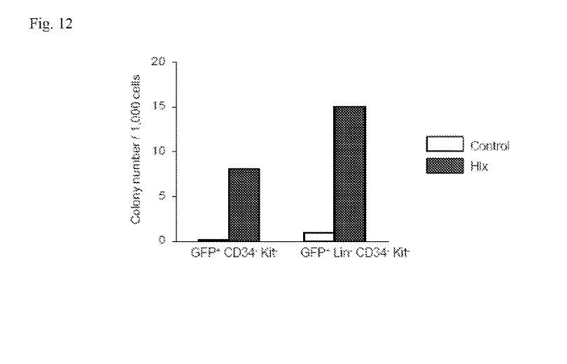

To investigate the effect of Hlx overexpression with regards to differentiation capacity, the clonogenic GFP+CD34−Kit− cells from the primary colonies were analyzed for the expression of additional cell surface markers. Strikingly, the proportions of Gr1+Mac1+ and Gr1−Mac1+, as well as F4/80+Mac1+ expressing cells were significantly reduced, indicative of a defect in myelo-monocytic differentiation (FIG. 3A). At the same time, expression of erythroid, B-lymphoid, or T-lymphoid markers was unchanged (FIG. 3A). Interestingly, almost half of the Hlx-overexpressing GFP+CD34−kit− population was lineage (Gr−1, Ter119, F4/80, CD19, B220, CD3)-negative, whereas only 16% of GFP+CD34−Kit− cells from control-transduced cells were lineage-negative (FIG. 3A). When Hlx-overexpressing GFP+Lin−CD34−kit− cells were sorted and tested in colony formation assays, they also showed a significant increase in clonogenicity (FIG. 12), indicating that Hlx acts at the level of Lin−CD34−kit− cells. To specifically test myelo-monocytic differentiation, colony-formation assays were conducted with GM-CSF or M-CSF stimulation, respectively. Hlx-transduced cells gave rise to significantly lower numbers of Gr1−Mac1+ and F4/80+Mac1+ cells compared to control-transduced cells, upon either GM-CSF or M-CSF stimulation (FIG. 3B, D). Cytomorphological evaluation of cells after stimulation showed an increased percentage of Hlx-transduced cells with immature progenitor morphology, in stark contrast to control-transduced cells which predominantly displayed mature monocytic morphology (FIG. 3C, E). Taken together, these findings show that Hlx not only enhances clonogenicity of an increased population of Lin−CD34−Kit− cells, but also confers a partial myelo-monocytic differentiation block.

Hlx Downregulation Inhibits Acute Myeloid Leukemia

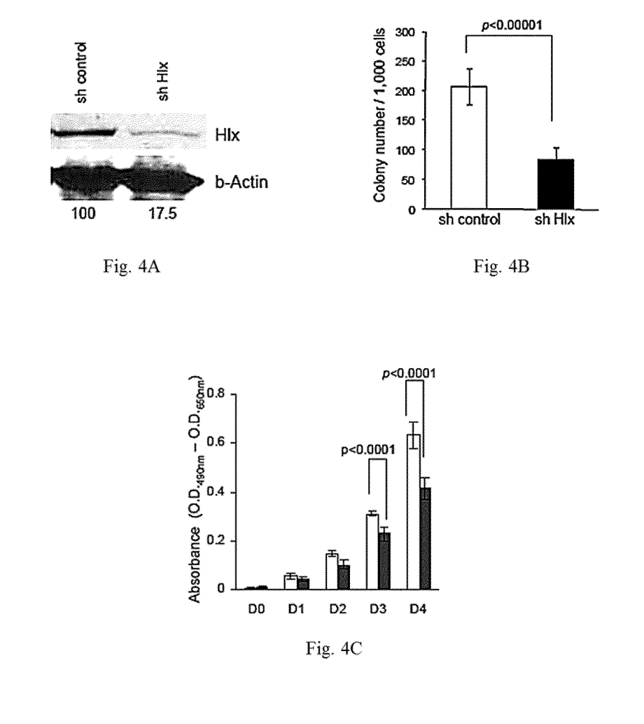

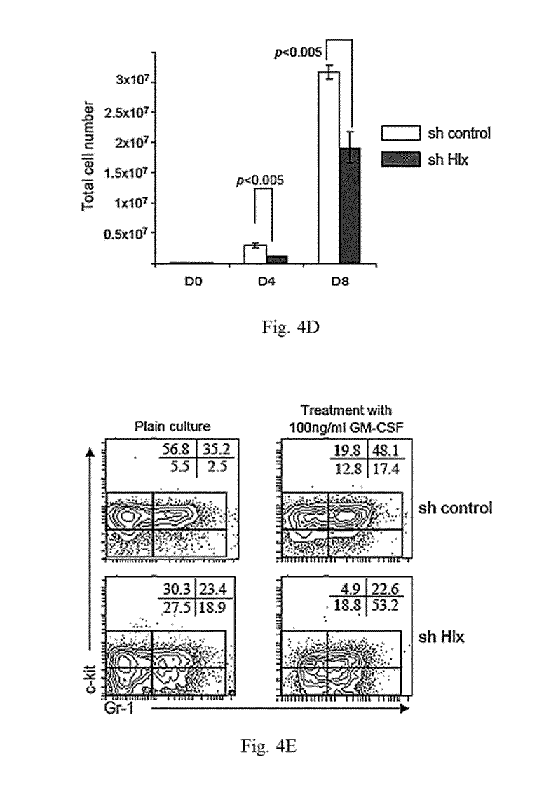

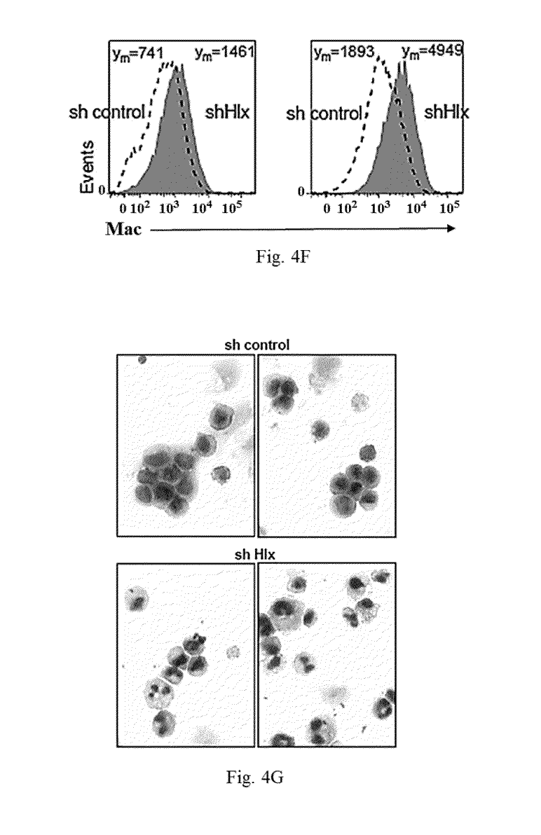

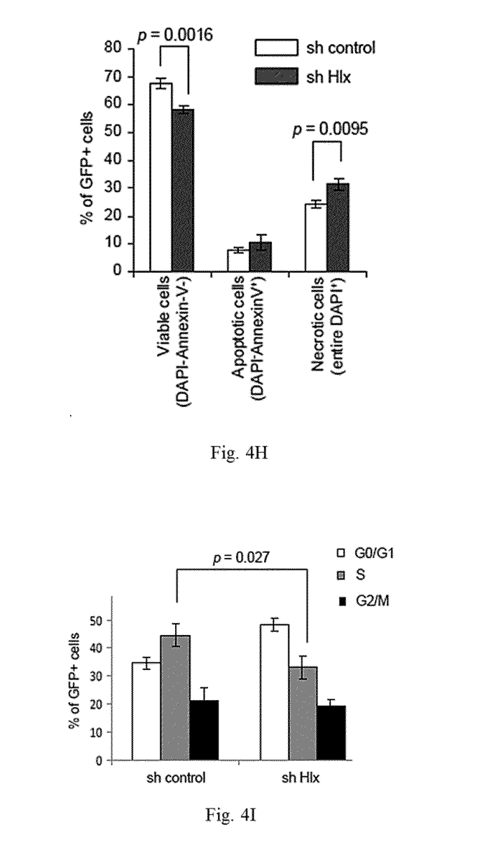

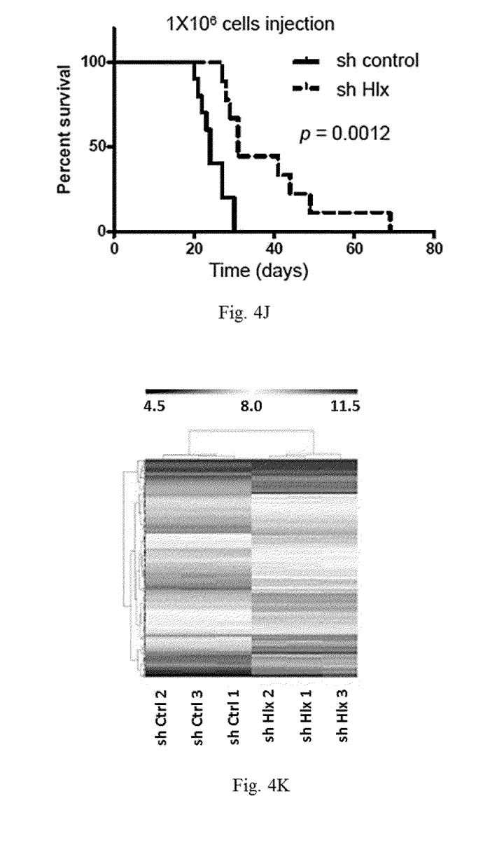

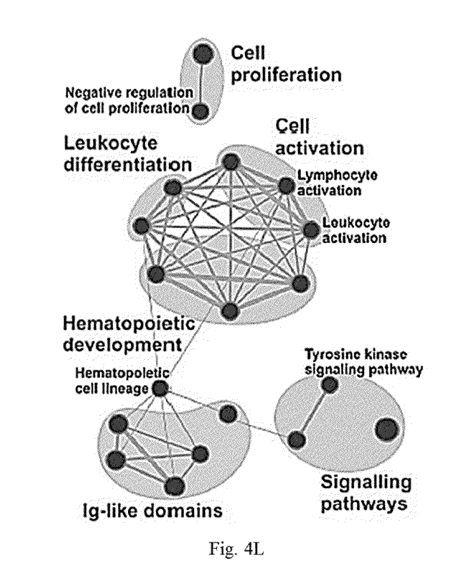

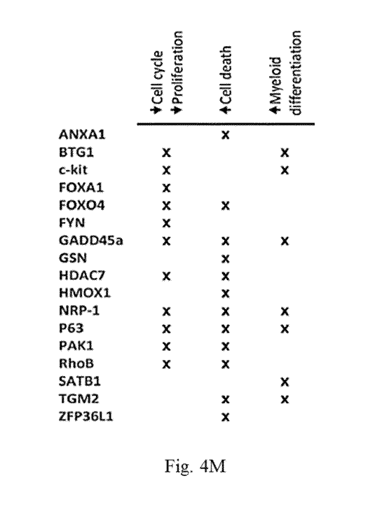

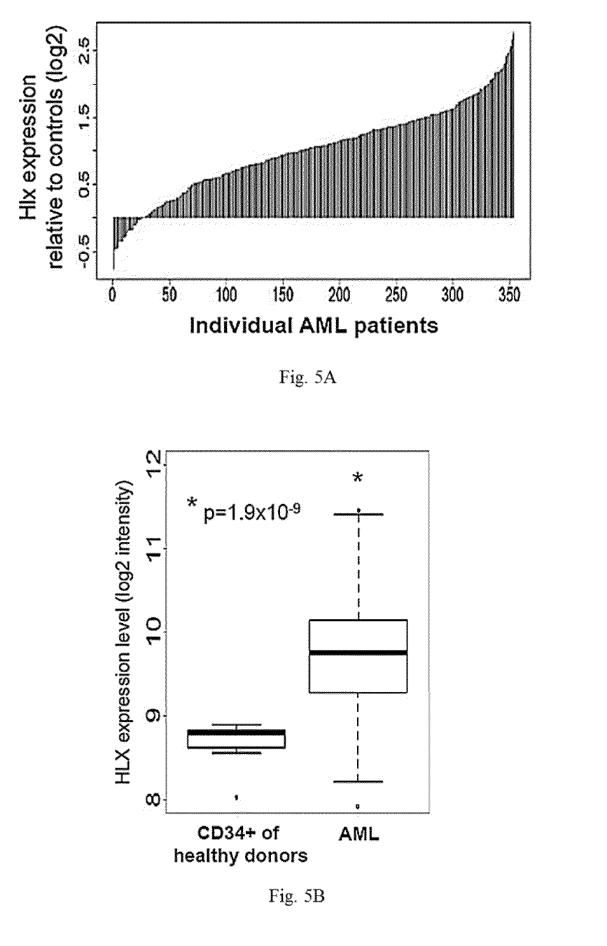

To test the hypothesis presented herein that Hlx overexpression is functionally important for acute myeloid leukemia cells a series of inhibition experiments were carried out utilizing RNA interference targeting Hlx. Leukemia cells derived from the PU.1 UREΔ/Δ AML model (URE cells; which express high levels of Hlx) were transduced with lentiviral constructs expressing either an Hlx-directed (sh Hlx) or a control shRNA (sh control). Strikingly, knockdown of Hlx by 80% led to significantly reduced formation of colonies of leukemic cells in methylcellulose assays in comparison to control-treated cells (median: 208 [SD; 30] colonies in sh control versus 85 [SD; 19] colonies in sh Hlx; p<0.00001) formation of leukemic colonies in methylcellulose assays in comparison to control-treated cells (FIG. 4A, B). Likewise, reduction of Hlx levels significantly reduced cell proliferation in suspension culture, as determined by MTS assays and manual cell counts (FIG. 4C, D). Examining the differentiation of the cells by cell surface markers, it was found that reduction of Hlx leads to an increased population of cells expressing lower levels of c-Kit and higher levels of Mac1, indicative of myeloid differentiation (FIG. 4E, F). Stimulation with GM-CSF further increased the number of Mac1 and Gr−1 expressing cells and cytomorphologically led to partial differentiation of acute myeloid leukemia cells in sh Hlx treated cells in comparison to control-treated cells, which retained an immature, leukemic morphology (FIG. 4G). Viability staining with Annexin V/DAPI indicated that Hlx downregulation in URE cells led to a statistically significant decrease in viable cells, and an increase in necrotic cells (FIG. 4H). This was also accompanied by a lower number of cells in S phase, and a higher number of cells in G1 phase of cell cycle (FIG. 4I). To test the anti-leukemic effect of Hlx downregulation in vivo, murine transplantation assays of cells transduced with either Hlx-directed or control shRNAs were performed. Strikingly, it was found that reduction of Hlx levels in transplanted URE cells prolonged recipient animal survival in comparison to mice transplanted with control shRNA-transduced cells (p=0.0012) (FIG. 4J). Taken together, the findings demonstrate that targeting Hlx can rescue the myeloid differentiation block in acute myeloid leukemia, inhibit growth and decrease clonogenicity, and lead to improved survival in a murine transplantation model.

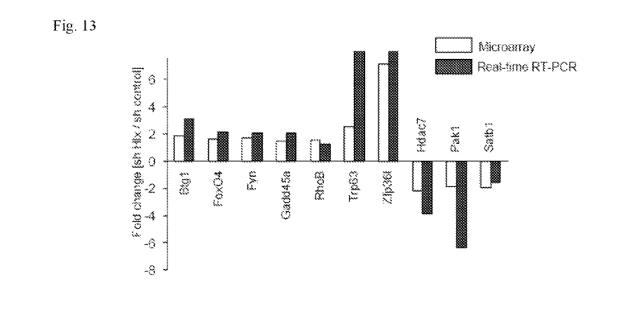

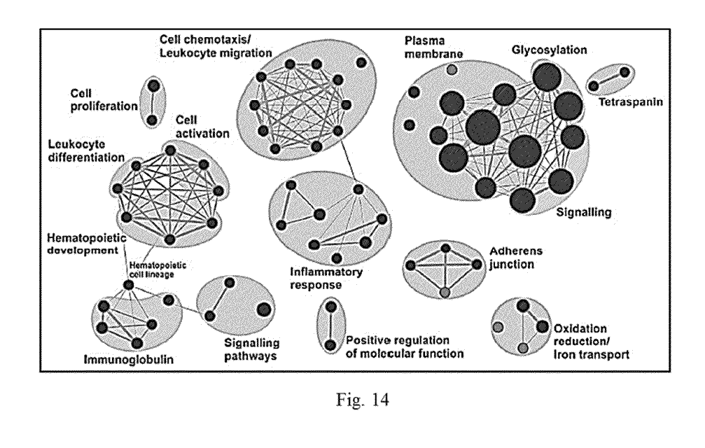

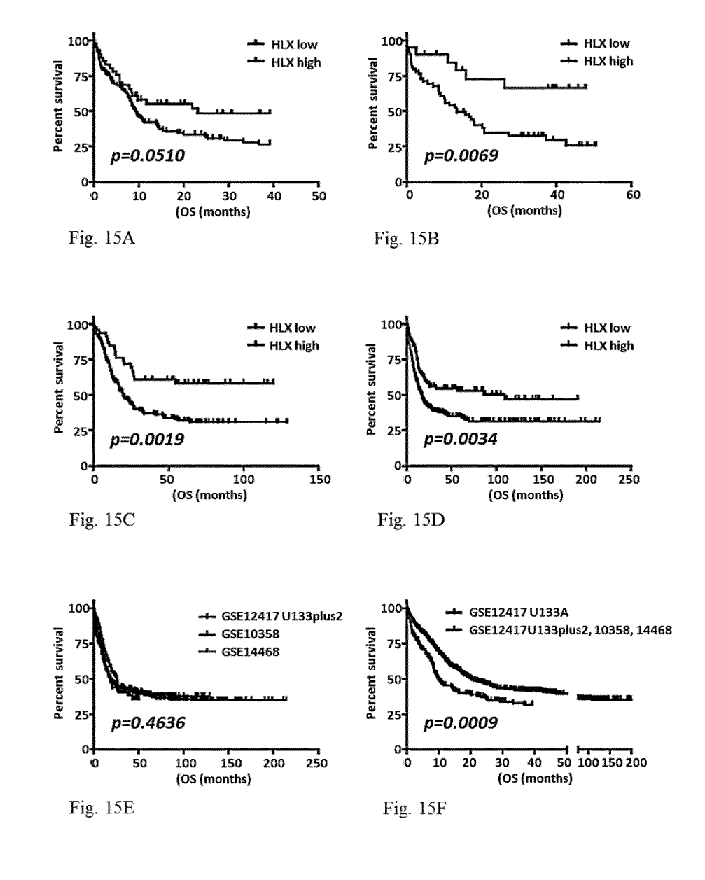

To gain insight into the molecular effects caused by Hlx inhibition gene expression profiles of shHlx-transduced URE cells and control shRNA-transduced cells were measured. Leukemia cells treated with Hlx-directed shRNAs displayed markedly different gene expression patterns with 392 genes being significantly differentially expressed (FIG. 4K). Gene set enrichment analysis showed that “cell lineage commitment”, “cell differentiation”, “cell activation”, and “cell proliferation” were among the most significantly affected cellular functions (FIG. 4L). These gene expression changes are highly consistent with the leukemia-inhibitory effect of Hlx reduction in URE cells. Several key genes involved in the regulation of cell cycle and proliferation, cell death, and myeloid differentiation, were significantly changed upon Hlx downregulation (FIG. 4M). Differential expression of several genes, namely Btg1, FoxO4, Gadd45a, Tp63, Hdac7, Pak1, and Satb1 was confirmed by quantitative real-time PCR (FIG. 13). Enrichment of genes involved in pathways of other cellular functions was also found including leukocyte migration, plasma membrane composition, and inflammatory response (FIG. 14). Further, gene set enrichment analysis (GSEA) software was utilized to compare the Hlx knockdown data with the molecular signatures database (MSigDB) [Ref. Subramanian, Tamayo, et al. (2005, PNAS 102, 15545-15550) and Mootha, Lindgren, et al. (2003, Nat Genet 34, 267-273)]. Significant negative enrichment of several known leukemia- and stem cell-related gene signatures was found in a gene set enrichment analysis of gene signatures from experimental models with altered Hlx expression. The data showed correlation of Hlx levels with several leukemia and stem cell gene signatures. Hlx knock-down in URE cell line and Hlx overexpression in sorted murine c−kit+ Sca−1+ lineage− (CD4− CD8− Ter119− B220− CD19− Gr1−) cells was investigated. Enrichment and normalized enrichment scores were determined Taken together, these data are consistent with a model that Hlx overexpression leads to activation of a specific transcriptional program in leukemia cells which affects processes critical for leukemogenesis such as cell differentiation and proliferation, and which can at least partially be reversed by inhibition of Hlx for therapeutic purposes.

HLX is Overexpressed in Patients with Acute Myeloid Leukemia

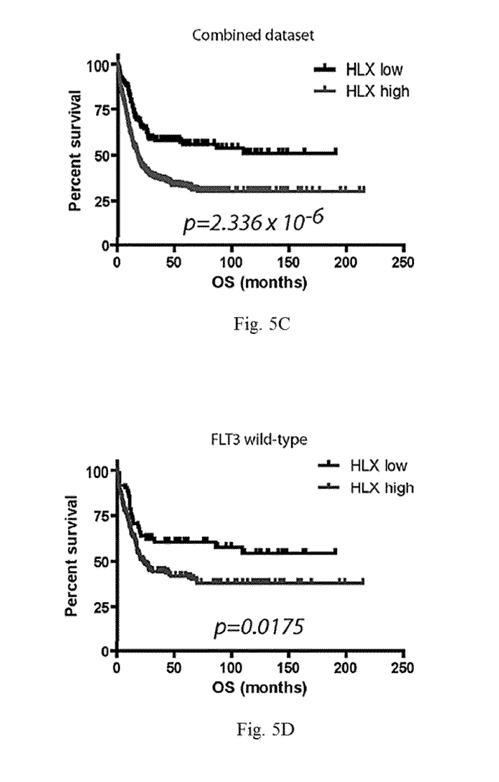

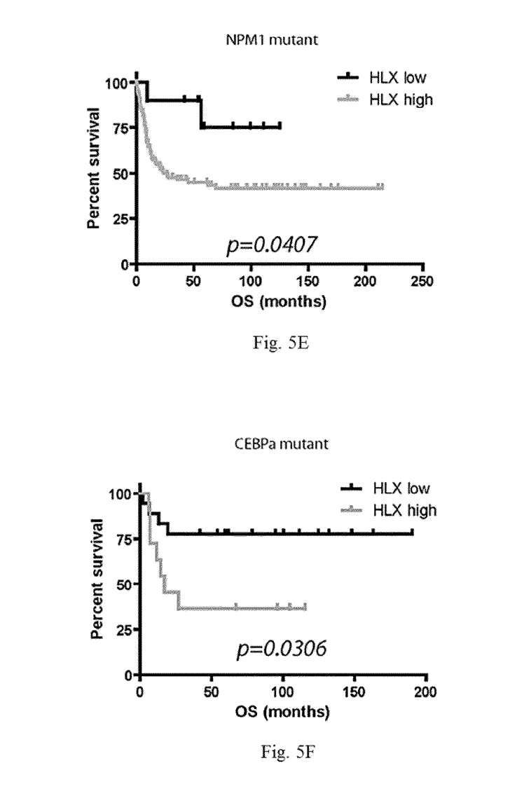

To examine whether HLX overexpression plays a role in human leukemia, gene expression data of 344[U1] patients with acute myeloid leukemia (Figueroa et al., Cancer Cell, January 2010) was analyzed. Strikingly, HLX was overexpressed in the majority of patients with AML in comparison to CD34+ cells of healthy donors (FIG. 5A). Overall, the average of HLX expression was 2.03-fold higher in AML patients (FIG. 5B), and this was statistically highly significant (p=1.9×10-9). 54% (185 out 344) of patients with AML overexpressed HLX more than 2-fold, and 25% of patients displayed higher than 2.73-fold overexpression with the range extending up 6.8-fold overexpression. These results demonstrate that HLX overexpression is a common feature in patients with AML.

Increased HLX Expression Correlates with Inferior Survival

Whether HLX expression levels in patients were associated with any known clinical or molecular parameters was examined. For that purpose 4 published datasets of patients with AML, of whom gene expression and time-to-event data were available (GSE10358, GSE12417 (U113A), GSE12417 (U133plus2), GSE14468), were analyzed. As the lower 25% of patients had HLX expression levels very similar to CD34+ cells of healthy donors (FIG. 5B), the 25th percentile was used to dichotomize patients into “HLX high” and “HLX low” expressers. The overall survival of AML patients was compared with low versus high HLX, and it was observed that in each of the 4 different data sets, high levels of HLX expression were associated with inferior overall survival (FIG. 15A-D). Overall survival (irrespective of HLX status) in datasets GSE12417 (U133plus2.0), GSE14468 and GSE10358 was very similar, with superimposable survival curves (p=0.4636, log-rank test; FIG. 15E, 15F), suggesting that the patient populations in these datasets and their clinical outcomes were comparable and could be combined for further analyses. Consistent with the analyses of the individual datasets, the evaluation of the combined set of patients from the GSE10358, GSE12417 (U133plus2.0) and GSE14468 datasets (N=601 total) confirmed that high HLX levels are associated with inferior overall survival (p=2.336×10-6 (log-rank); hazard ratio (HR)=0.57 (95% confidence interval: 0.046-0.71); median survival: 17.05 months for HLX high, not reached for HLX low; 5-yr survival rate: 32.95% for HLX high, 55.85% for HLX low) (FIG. 5C).

To assess whether the impact of HLX expression on overall survival is independent of known prognostic factors for AML, multivariate analysis was performed based on the data of the initial 344[U2] patients (Figueroa et al. Cancer Cell, January 2010), using a Cox regression model. In this analysis, high HLX status remained an independent prognostic factor (p=0.0416, HR 1.521) along with FLT3 mutation status (p=0.0003, HR 1.925), NPM1 mutation status (p=0.0006, HR 0.518), CEBPA mutation status (p=0.0371, HR 0.693), and cytogenetic risk group (p=0.0109, HR 1.382). The independent prognostic role of HLX status indicated that it may provide additional prognostic information for patients who belong to previously established, but prognostically heterogeneous, molecularly defined subtypes of AML. Indeed, it was observed that among patients in certain molecularly defined subtypes which are considered prognostically favorable, namely FLT3 wild-type status, NPM1 mutation, or CEBPα mutation, high HLX expression is associated with inferior overall survival (p=0.0175, p=0.0407 and p=0.0306, respectively) (FIG. 5B-D).

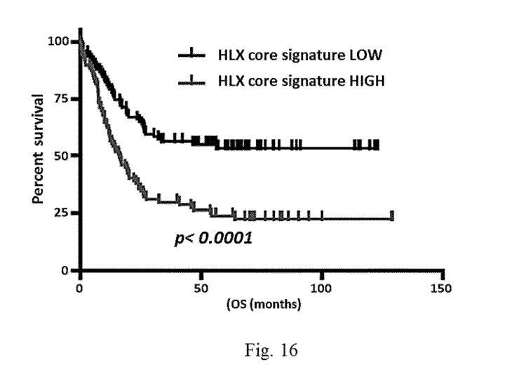

To gain insight into the molecular consequences of elevated Hlx levels, Hlx was overexpressed in sorted LSK cells and genome-wide transcriptional analysis was performed. It was found that 195 genes were significantly changed, resulting in a clearly distinguishable expression signature induced by Hlx overexpression. Data were analyzed as hierarchical clustering of genes differentially expressed upon Hlx overexpression in sorted LSK cells. Genes with −log 10 (p) value <0.1 and a group mean difference >0.5 (log 2 scale) were considered differentially expressed. After filtering out unannotated and duplicate genes, genes were clustered by hierarchical, Euclidean distance, complete linkage clustering. Using GSEA enrichment of known leukemia- and stem cell-related gene signatures was found, which is consistent with this laboratory's findings. Next, human “Hlx high” signatures were generated from each of the 3 gene expression data sets of patients with AML. Genes differentially expressed in patients with low versus high HLX were identified (lower bound of fold-change<1.0, p<0.05, FDR<10%[U3]) and a common signature across different AML datasets was generated by cross-comparison of individual signatures. This “Hlx high” signature in human AML was then overlaid with the signature obtained from the Hlx overexpression experiment. The resultant consensus “Hlx core signature” comprised a total of 25 genes, each of which were commonly changed in all AML data sets and upon Hlx overexpression in LSK cells (Table 1). To test if the Hlx core signature was clinically relevant, patients were dichotomized based on their overall score into “Hlx core signature LOW” and “Hlx core signature HIGH”. When the overall survival of AML patients was compared, it was observed that patients of the “Hlx core signature HIGH” group showed strikingly inferior overall survival (FIG. 16) (p<0.0001 (log-rank), HR=XX), with a median survival of 15.5 months (versus “not reached”, in HLX core signature LOW patients), 5 year overall survival of 23% (versus 53%).

TABLE 1
HLX core signature

MPEG1 macrophage expressed gene 1
CD86 CD86 molecule
CLEC4A C-type lectin domain family 4, member A
ITGB2 Integrin, beta 2 (complement component 3 receptor 3 and 4
subunit)
CD93 CD93 molecule
FCER1G Fc fragment of IgE, high affinity I, receptor for; gamma
polypeptide
CSF1R colony stimulating factor 1 receptor
RAB31 RAB31, member RAS oncogene family
ITGAM integrin, alpha M (complement component 3 receptor 3 subunit)
TMEM71 transmembrane protein 71
IL17RA interleukin 17 receptor A
RAB31 RAB31, member RAS oncogene family
SCPEP1 serine carboxypeptidase 1
S100A4 S100 calcium binding protein A4 (calvasculin, metastasin)
CD300A CD300a molecule
FGL2 fibrinogen-like 2
AHNAK AHNAK nucleoprotein (desmoyokin)
IFNGR1 interferon gamma receptor 1 /// interferon gamma receptor 1
ATP8B4 ATPase, Class I, type 8B, member 4
IFNGR2 interferon gamma receptor 2 (interferon gamma transducer 1)
LYZ lysozyme (renal amyloidosis)
B3GNT5 UDP-GlcNAc: betaGal beta-1,3-N-acetylglucosaminyltransferase
5
RGS2 regulator of G-protein signalling 2, 24 kDa
AIF1 allograft inflammatory factor 1
RBMS1 RNA binding motif, single stranded interacting protein 1

For the generation of the Hlx signature, the RMA-normalized log 2-transformed GSE14468 gene expression data were dichotomize into HLX low (lower 25th percentile of Hlx expression) and HLX high (the remaining samples) sets, as done for the survival analysis, and SAM analysis was performed to identify differentially expressed genes between these groups. This list of genes was subsequently intersected with the human orthologs of the genes that were differentially expressed in the mouse overexpression or knockdown models, and which showed the same directionality of expression differences relative to Hlx levels between data from the Hlx knockdown or overexpression experiments with murine cells and human data. This list of 45 genes was subsequently used as covariates for Hlx expression and for overall survival in the R/Bioconductor globaltest function (Goeman, van de Geer, et al, 2004), and the most significantly correlated genes (17) were selected to define an Hlx-associated signature (“Hlx signature”). [C4]

To calculate a signature score, expression of each gene was median-centered to give equal weight to each component of the signature, and the mean of the positively associated minus the mean of the negatively associated genes was calculated for each patient sample. The samples from GSE14468 (test set) and GSE10358 (validation set) were then ranked and dichotomized according to this normalized signature score.[C5] A score can be calculated and preferably the directionality is factored in. The negatively associated genes should be factored in with a “minus”, so that they further enhance the score. In an embodiment of the methods described herein, the subject is assessed by determining the signature score and confirming if the signature score is above a predetermined signature score (e.g. from a control).

The individual genes included in the HLX signature are as follows (3 negatively associated; 14 positively associated):

ZNF451 (negative)
AIG1 (negative)
GALC (negative)
PGD (positive)
RASGRP4 (positive)
ITGAM (positive)
PAK1 (positive)
CD53 (positive)
GCH1 (positive)
GADD45B (positive)
NCOR2 (positive)
SFXN3 (positive)
PDLIM2 (positive)
AIF1 (positive)
PARVG (positive)
ZAK (positive)
IBRDC1 (positive)

### DISCUSSION

Utilizing both mouse and human systems it has been shown that the class II homeobox protein Hlx affects hematopoietic stem cell function, as well as clonogenicity and differentiation of immature hematopoietic progenitor cells. Furthermore, it was found that Hlx is significantly overexpressed in the majority of patients with acute myeloid leukemias, and that high Hlx expression levels are associated with inferior clinical outcome. Thereby, this study identifies Hlx as a novel class II homeobox gene which is critically involved in the pathogenesis of acute myeloid leukemia. The finding that increased Hlx expression correlates with more aggressive disease, combined with the observation that Hlx knockdown results in an inhibition of growth and clonogenicity of leukemia cells further shows that Hlx is a novel prognostic and therapeutic target.

Many clustered (class I, or HOX) homeobox genes have been implicated in normal hematopoiesis as well as leukemia, but much less is known about the role of non-clustered (class II) homeobox transcription factors (for review see Argiropoulos, Humphries, Oncogene 2007). Several HOX genes are expressed at high levels in subtypes of AML (Alcalay M, Blood 2005; Ayton, Cleary, Genes Dev 2003; Horton S J, Cancer Res 2005, Bullinger, NEJM 2004). Important roles in leukemic transformation have been demonstrated specifically for several members of the HOX-A and the HOX-B cluster (Sauvageau G, Immunity 1997; Thorsteinsdottir U, MCB 1997; Kroon, EMBO J 1998; Fischbach NA, Blood 2005; Krivtsov et al., Nature 2006; Somervaille, Cleary, Cancer Cell 2006). Also, the non-clustered homeobox gene CDX2 was recently reported to be implicated in leukemogenesis (Scholl C et al., J Clin Invest 2007). However, the clinical significance of these known HOX genes is largely unclear. Here, it is reported for the first time that levels of a homeobox gene is strongly associated with inferior overall survival in several large, independent cohorts of patients with AML. Furthermore, the prognostic value of HLX is a broad phenomenon across several molecular subsets of patients, and HLX holds up as an independent prognostic factor in a multivariate model. Gene expression analyses demonstrated that Hlx regulates the expression of a specific subset of genes and that this “Hlx signature” is also able to discriminate between patients with poor and favorable clinical outcome. Taken together, these observations suggest that HLX is a key regulator of a gene subset critical for AML pathogenesis, and that it defines a previously unrecognized molecular subtype of AML with distinct biological features and clinical outcome.

Several HOX genes such as Hoxb4 have been reported to be stimulators of HSC function and expansion (Savageau G, Genes Dev 1995; Antonchuk, Humphries, Cell 2002). Our data show that Hlx actually suppresses the function of normal immature HSC and progenitors, but leads to an increase of clonogenicity and a differentiation block at the level of phenotypically more mature progenitors. As the loss of HSC does not seem to be mediated by induction of apoptosis or necrosis, one may speculate that Hlx exerts this dual role by triggering initial differentiation of HSC and suppression of terminal differentiation at a more committed progenitor level. Further studies will be required to understand the molecular basis of this effect. Like other homeobox genes, Hlx may possibly function in concert with co-factors (Pineault N, MCB 2004; Moens and Selleri, Dev Biol 2006). Such co-factors could confer cell type specificity to the effects of Hlx overexpression, and also contribute to leukemic transformation.

Several transcription factors that govern normal hematopoietic differentiation have been implicated in leukemogenesis by blocking differentiation and promoting self-renewal and clonogenicity (for review see Tenen D G, Nat Rev Cancer 2003). Hlx may act similar to those factors by establishing a specific gene expression program in committed progenitors, which results in increased long-term clonogenicity and a differentiation arrest, and also contributes to poor clinical outcome. Thus, Hlx expression levels may be utilized to predict clinical outcome and improve risk stratification. Furthermore, inhibition of Hlx may be a novel promising strategy for treatment of patients with acute myeloid leukemia.

### Methods and Materials

Mice and Cells

FVB/nJ mice (Ly5.1), C57BL/6J (Ly5.2) mice, and B6.SJL-Ptprca Pepcb/BoyJ (Pep boy, Ly5.1) mice were used for in vitro assays and in vivo transplantation assays. NOD.Cg-Prkdcscid Il2rgtm1Wj1/SzJ (NSG) mice were used for in-vivo transplantation assays using leukemia cells. PU.1-knockdown mice with targeted disruption of the distal enhancer (URE) −14 kb upstream of the PU.1 gene have been previously described (Rosenbauer 2004). All animal experiments were performed in compliance with institutional guidelines and approved by the Animal Institute Committee of the Albert Einstein College of Medicine (protocol #20080109). URE cells were established as described previously and maintained in M5300 media (Stem Cell Technologies) supplemented with 10% heat-inactivated FBS, 15% supernatant of WEHI-3B culture medium, 15% supernatant of BHK culture medium and penicillin/streptomycin [Steidl 2006].

Flow Cytometric Analysis and Sorting

Mononuclear cells were purified by lysis of erythrocytes before analyzing BM or PB. For analysis and sorting antibodies we used directed against CD4[GK1.5], CD8a[53-6.7], CD19[eBio1D3], Gr-1[RB6-8C5], B220[RA3-6B2], F4/80[BM8], c-kit[ACK2], Sca-1[D7], CD34[RAM34], CD16/32[93], CD150[TC15-12F12.2], CD48[HM48-1], Flk-2[A2F10], Mac1[M1/70], Ter119[TER-119], and Thy-1.2[53-2-1]. To distinguish donor from host cells in transplanted mice, cells were additionally stained with anti-CD45.1[A20] and CD45.2[104]. Analysis and sorting were performed using a FACSAria II Special Order System (BD Biosciences, San Jose, Calif.). For sorting Lin−Kit+ cells for in vivo assay, TRI-color or PE-Cy5-conjugated CD4, CD8a, CD19, B220, Ter119, and Gr-1 anti-lineage antibodies were used, and APC-conjugated c-kit antibody. For analyzing hematopoietic stem and early progenitor cells, PE-conjugated Ly5.2 antibody, PE-Cy5-conjugated CD4, CD8a, CD19, B220 and Gr-1 anti-lineage antibodies, APC-conjugated c-kit antibody, pacific blue-conjugated Sca-1 antibody, PE-Cy7-conjugated Thy1.2 antibody, and biotin-conjugated Flk-2 antibody followed by APC-AlexaFluor 750 conjugated streptavidin was used. For analyzing committed progenitors APC conjugated Ly5.2 antibody, PE-Cy5-conjugated CD4, CD8a, CD19, B220 and Gr-1 anti-lineage antibodies, APC-AlexaFluor 780-conjugated c-kit antibody, pacific blue-conjugated Sca-1 antibody, PE-conjugated FcγRII/III antibody, and biotin-conjugated CD34 antibody followed by PE-Cy7-conjugated streptavidin was used. For differentiation studies, PE conjugated GR-1 antibody, APC conjugated Mac1 antibody, eFluorTM450 conjugated F4/80 antibody and APC-AlexaFluor 780-conjugated c-kit antibody were used.

Lentiviral Vectors and Transduction

For overexpression studies, an Hlx-expressing lentivirus was created by introducing the mouse Hlx coding sequence into the EcoRI site of a pCAD-IRES-GFP lentiviral construct (Steidl 2007). For knockdown studies, shRNA template oligonucleotides (target sense strand-loop-target antisense strand-TTTTT, luciferase target (gtgcgttgttagtactaatcctattt) were inserted as a control or mouse Hlx target (ggcgcagaaggacaaggacaaggaagcgg) for Hlx knockdown into the pSIH1-H1-copGFP shRNA vector (System Biosciences, Mountain View, Calif.). For production of lentiviral particles, lentiviral constructs were transfected with packaging vectors into 293T producer cells, harvested supernatant after 48 and 72 hours, and concentrated by ultracentrifugation. For overexpression studies, sorted Lin−Kit+ cells from wild-type C57BL/6J (Ly5.2) bone marrow (for in vivo assay) or Lin−Kit+Sca-1+ cells from wild-type FVB/nJ bone marrow (for in-vitro assay) were treated with control virus (IRES-GFP) or Hlx virus (IRES-GFP-Hlx). Briefly, sorted cells were cultured in Iscove's modified Dulbecco's medium (IMDM) containing heat-inactivated FBS, mIL-3, mIL-6 and mSCF with lentiviral supernatants in the presence of 8 μg/ml polybrene. 24 hours after transduction, cells were washed with PBS and then used for experiments. 40 hours after transduction, the efficiency of transduction was analyzed by checking the frequency of GFP-positive cells by flow-cytometry. For knockdown studies, cells were incubated with short-hairpin-containing lentivirus for 24 hours. After culture with fresh medium, GFP-positive cells were sorted using a FACS Aria II sorter (BD Biosciences) and used for experiments.

Quantitative Real-Time PCR

Total RNA was extracted from FACS-sorted cells or cultured cells using RNeasy Micro kit (Qiagen, Valencia, Calif.) and then synthesized cDNA by Superscript II reverse transcriptase (Invitrogen, Carlsbad, Calif.). Real-time PCR was performed using an iQ5 real-time PCR detection system (BIO-RAD, Hercules, Calif.) with 1 cycle of 50° C. (2 min) and 95° C. (10 min) followed by 40 cycles of 95° C. (15 sec) and 60° C. (1 min) using Power SYBR Green PCR master mix (AB, Carlsbad, Calif.) (for primer sequences, see Table 2). Measurements were quantified using the ΔΔCT method, normalized to Gapdh, and expressed relative to the expression in indicated calibrators.

TABLE 2
Primers for real-time PCR (SEQ ID NOs. 2-19,
respectively).

| | |
|---|---|
| mouse Hlx FW | TTCAGCATCAATTCCAAGACACA |
| mouse Hlx RV | ACCTCTTCTCCAGGCCTTTTCT |
| mouse Btg1 FW | TCATCTCCAAGTTCCTCCGCAC |
| mouse Btg1 RV | CAACGGTAACCTGATCCCTTGC |
| mouse FoxO4 FW | TCTACGAATGGATGGTCCGCAC |
| mouse FoxO4 RV | CTTGCTGTGCAAGGACAGGTTG |
| mouse Gadd45a FW | CCTGGAGGAAGTGCTCAGCAAG |
| mouse Gadd45a RV | GTCGTCTTCGTCAGCAGCCAG |
| mouse Hdac7 FW | CGCCTCAAACTGGATAACGGGA |
| mouse Hdac7 RV | GCATTGGAGGAATGCAGCTCGT |
| mouse Pak1 FW | ATTGCTCCACGCCCAGAACACA |
| mouse Pak1 RV | AAGCATCTGGCGGAGTGGTGTT |
| mouse Satb1 FW | CGCCATTGAATATGATTGCAA |
| mouse Satb1 RV | TCCAACCTGGATTAGCCCTTT |
| mouse Gapdh FW | CCAGCCTCGTCCGTAGAC |
| mouse Gapdh RV | GCCTTGACTGTGCCGTTGA |
| mouse Trp63 FW | TGAGCCGTGAGTTCAATGAG |
| mouse Trp63 RV | ACCTGTGGTGGCTCATAAGG |

Western Blotting

Total cell lysates were extracted in lysis buffer (50 mM Tris-Cl (pH7.5), 1 mM EDTA, 150 mM NaCl, 1% NP-40, 1% sodium deoxycholate, 1 mM PMSF, and protease inhibitor cocktail (Roche)). Anti-Hlx polyclonal rabbit antibody (SantaCruz, clone H-130, sc-135014) and anti-actin polyclonal goat antibody (Santa Cruz, clone C-11, sc1615) were used as primary antibodies, HPRT-conjugated anti-rabbit or anti-goat antibody (Santa Cruz) were used as secondary antibodies. ECL solution (Pierce) was used for detection of bands.

Cell Proliferation Assays

For MTS assays, 1×10⁴ cells per well were plated into 96-well plates with 100 μL culture medium. After incubation with 20 ul of MTS reagent (CellTiter 96® AQueous One Solution Cell Proliferation Assay kit, Promega), OD490 and OD650 were detected by a microplate reader (Versa max, Molecular probe). Raw values were compensated by subtraction of background, defined as [OD490-OD650] of a well with cells minus [OD490-OD650] of a well with medium only. Manual cell counts were performed culturing 1×10⁵ cells per well in 24-well plates with 1 ml medium. Viable cells were counted using trypan blue exclusion and re-adjusted to 1×10⁵ cells per well every 4 days.

Cell Cycle Assays

The Click-iTTM EdU Flow Cytometry Assay system (Invitrogen, Life Technologies) was used following the manufacturer's instructions. Briefly, after culture of cells with EdU (10 μM) for 2 hours, cells were fixed by 4% paraformaldehyde, treated with saponin containing buffer, and then incubated with Alexa Fluor 647 dye azide. DAPI was added directly before flow cytometric analysis.

Apoptosis Assays

Apoptotic and necrotic cells were analyzed by use of Annexin V/DAPI staining as previously described (Kawahara Blood 2008). Briefly, cells were treated with PE-AnnexinV (BD Pharmingen) and DAPI in Ca2+ containing buffer. Then cells were analyzed by flow-cytometry.

Colony Formation Assays and Serial Replating Assays

To investigate clonogenic capacity of lentivirus-transduced cells, these assays were performed in MethoCult M3434 (Stem Cell Technologies, Vancouver, BC) containing IL-3, IL-6, SCF, and EPO or in MethoCult M3234 supplemented with M-CSF or GM-CSF as previously described [Cozzio, Huntly, Steidl]. GFP-positive colonies were scored 8-10 days after plating lentivirus-transduced cells using an AXIOVERT 200M microscope (Zeiss, Maple Grove, Minn.). After the first plating/scoring, we re-sorted GFP-positive cells and then proceeded with serial replating assays. Cells were replated in M3434 MethoCult and GFP-positive colonies were again scored after 10-14 days.

Transplantation Assays

For Hlx overexpression studies, 5×104 lentivirus-transduced Lin−Kit+ cells (Ly5.2) together with 2.5×105 spleen cells from congenic wild-type recipients (Ly5.1) were transplanted into lethally irradiated age-matched congenic wild-type recipients (Ly5.1) by retroorbital vein injection. Peripheral blood was analyzed 8 weeks and 12 weeks after transplantation. At 12 weeks, recipient mice were sacrificed and bone marrow was analyzed. Total body irradiation was delivered in a single dose of 950 cGy using a Shepherd 6810 sealed-source 137Cs irradiator.

Micorarray Experiments and Analysis

RNA was extracted from sorted GFP-positive cells utilizing the RNeasy Micro Kit (Qiagen). After evaluation of the quality of RNA with an Agilent2100 Bioanalyzer, total RNA was used for amplification utilizing the Nugen Ovation pico WTA system according to the manufacturer's instructions. After labeling with the GeneChip WT terminal labeling kit (Affymetrix), labelled cRNA of each individual sample was hybridized to Affymetrix Mouse Gene 1.0ST microarrays (Affymetrix), stained, and scanned by GeneChip Scanner 3000 7G system (Affymetrix) according to standard protocols. The complete array data is deposited in the gene expression omnibus (Edgar et al., 2002) and are accessible through GEO series accession number GSE27947 (www.ncbi.nlm.nih.gov/geo/query/acc.cgi?acc=GSE27947). Raw data was normalized with the RMA algorithm of Affymetrix Power Tools v. 1.178 using the following parameters: apt-probeset-summarize −a rma −b MoGene-1_{—}0-st-v1.r4.bgp −c MoGene-1_{—}0-st-v1.r4.clf −m MoGene-1_{—}0-st-v1.r4.mps—qc-probesets MoGene-1_{—}0-st-v1.r4.qcc −p MoGene-1_{—}0-st-v 1.r4.pgf −o APT—summaries—cel-files cel_list.txt. A t-test with Welch approximation for unequal group variances with p-values based on t-distribution was performed with a cutoff of p<0.05 (Hlx knockdown experiment) or p<0.1 (Hlx overexpression experiment) in Multiple Experiment Viewer v.4 pilot2 (www.tm4.org/mev/) [Ref PMID: 16939790]. Subsequently, probes with −log 10(p) value <0.05 (or <0.1 for overexpression experiments) and a group mean difference >0.5 (log 2 scale) were considered differentially expressed and used for further analysis. After filtering out unannotated and duplicate genes, the remaining genes were clustered by hierarchical clustering, with optimization of sample and gene leaf order, using Euclidean distance, complete linkage clustering. For enrichment map analysis, gene enrichment table files were generated using the DAVID bioinformatics tool, filtered for significance with p-value and FDR thresholds set at <0.05 and <0.25, respectively, and visualized using the Enrichment Map Cytoscape plugin. The gene lists were also analyzed by Gene Set Enrichment Analysis v2.0 (GSEA) (Subramanian, Tamayo, et al. (2005, PNAS 102, 15545-15550) and Mootha, Lindgren, et al. (2003, Nat Genet 34, 267-273)), using gene set size filters of min=8, max=500, the permutation type set to gene_set, MSigDB v3.0 gene sets (c2.cgp.v3.0.symbols.gmt) and a cutoff at p<0.05.

Statistical Analysis

The publicly available gene expression data sets with accession numbers GSE12417 (training set in U133A and U133B; test set in U133plus2.0), GSE14468, and GSE10358 (available at Gene Expression Omnibus (GEO) Database, www.ncbi.nlm.nih.gov/geo/) were analyzed. Clinical outcome and mutational data for the GSE10358 dataset were obtained from a recent study of the same group (Ley, NEJM 2010). Analyses of the gene expression profiles from GSE14468, GSE12417 training set and GSE10258 were performed based on published (Gentles A J, JAMA 2010) and publicly available MASS files (available in GEO entry GSE24006) with reanalyzed data. For analysis of the test set of the GSE12417 dataset, CEL files were downloaded from GEO, and processed using GenePattern (Broad Institute, Cambridge Mass.) for normalization (ExpressionFileCreator algorithm) according to the preset parameters of the software (RMA method, with quintile normalization, background correction, median scale normalization method). All aforementioned datasets were then analyzed separately to dichotomize the population of patients of each dataset into subsets with high versus low expression of HLX transcript, using the 25th percentile of normalized HLX expression in each data set as the cutoff point. Publicly available clinical annotation accompanying each one of these data sets was then used to perform Kaplan-Meier survival analysis (GraphPad Prism 5.0) comparing clinical outcome of patients with high versus low HLX expression. Results were re-run using different methods of normalization and using different methods of calculation of the 25th percentile of HLX expression (e.g. for those datasets without available time-to-event data for some patients, repeat analysis were performed based on recalculation of the 25th percentile of HLX expression among only patients for who, overall survival information was available) and results were qualitatively consistent. Multivariate analyses using Cox regression models were performed (with Forward Conditional and Backward Conditional methods in the SPSS 18.0 statistical package) using the cytogenetic risk data and mutational status information available for patients from the GSE14468 dataset (including parameters such as age (< or >60 years old), gender, cytogenetic risk group, mutational status for FLT3ITD, FLT3D835 (TKD), NPM1, CEBPA, IDH1, IDH2, N-Ras, K-Ras, EVI1 expression and HLX status (low vs. high expression according to the 25th percentile cutoff point). Confirmatory multivariate analysis was performed in the data of the GSE10358 dataset for the clinical and molecular parameters available for that dataset.

Signature Generation

CEL files for publicly available gene expression datasets GSE12417 U133A, GSE12417 U133plus2, GSE10358 and GSE14468 were downloaded from the GEO database and processed separately for each dataset in dChip (biosun1.harvard.edu/complab/dchip/) for generation of DCP files. Data were then normalized and modeled according to the preset normalization parameters of the software (probe selection method: invariant set; smoothing method: running median). Patients in each dataset were characterized as having low or high HLX levels, using the 25th percentile of normalized signal for the HLX probe in each dataset as the dichotomization point. Genes differentially expressed in patients with low versus high HLX were identified using in dChip according the following criteria: ratio of average expression of >1.2 or <1.2 in patients with high vs low HLX; absolute difference in average signal in the 2 groups of >100; p-value <0.05; permutation testing (100 times) to asses false discovery rate (FDR) in each dataset. The 90th percentile of the number of probes with false discovery as part of this permutation testing was used as a cutoff to exclude from further analysis the probes with the highest p-value among those that satisfied the other comparison criteria. A common signature across different AML datasets was generated by cross-comparison of individual signatures, and this signature was then overlayed with signatures obtained from the Hlx overexpression experiment.

### Example 2

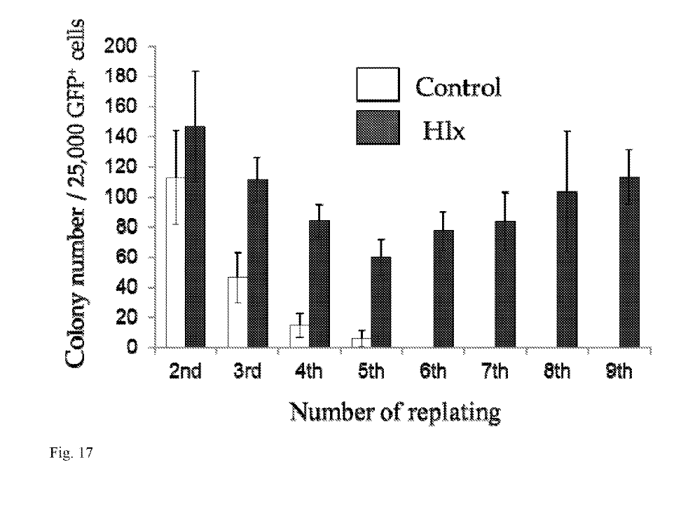

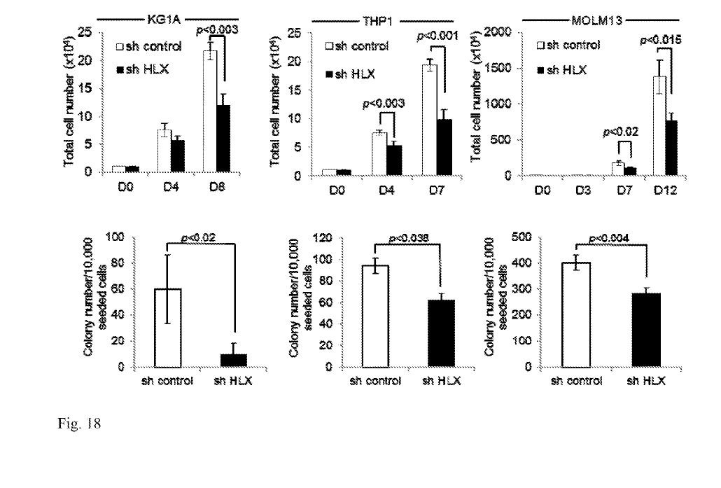

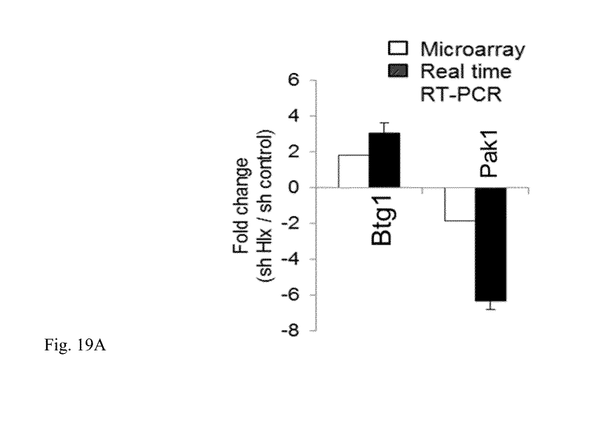

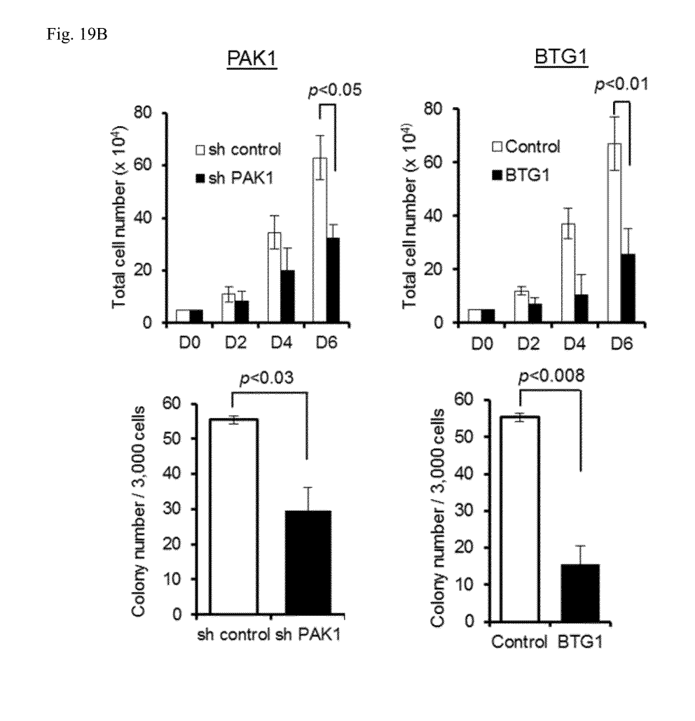

Further replating data was obtained showing “immortalization” of myeloid progenitors and unlimited clonogenicity with Hlx expression (see FIG. 17), and the inhibitory effect of HLX was demonstrated in several human AML cell lines (see FIG. 18). It was later discovered that Btg1 and Pak1 are functionally critical downstream genes of Hlx and mediate the anti-leukemic effect of Hlx inhibition (see FIG. 19). As such, Btg1 and Pak1 are therapeutic targets.

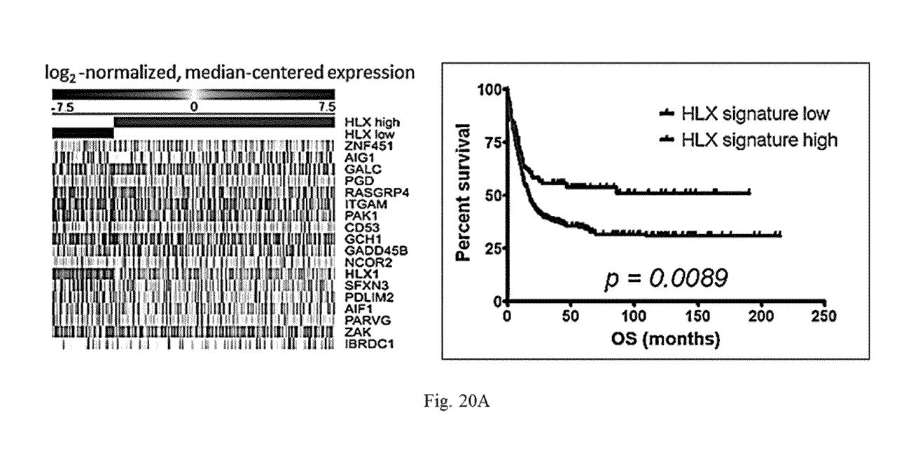

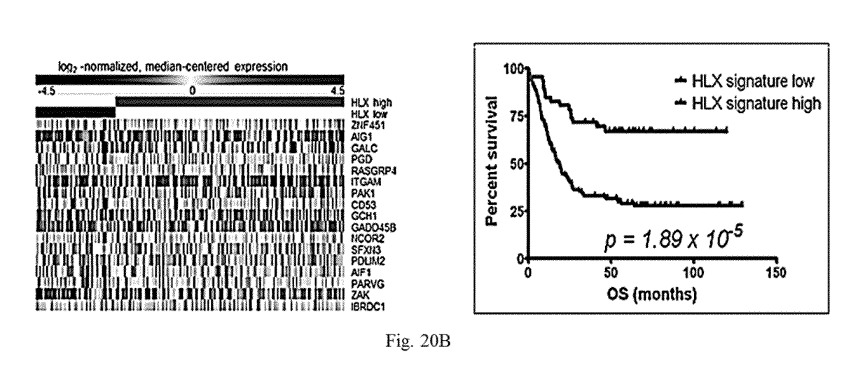

To obtain insight into the molecular consequences of elevated Hlx levels, Hlx was overexpressed Hlx in sorted murine LSK cells and a genome-wide transcriptional analysis performed. It was found that 195 genes were significantly changed, resulting in a clearly distinguishable expression signature induced by Hlx overexpression (data not shown). Next, it was tested if this mouse LSK Hlx overexpression gene set correlated with HLX expression in the human AML patient cohorts. Specifically, the human orthologs of the mouse gene set were compared to HLX expression levels of AML patients in the different cohorts using the globaltest package in R/Bioconductor (Goeman, van de Geer, et al, 2004). A highly significant correlation was found between the mouse gene signature and HLX expression in the human AML samples (p=7.43×10-23 for GSE14468, p=2.13×10-08 for GSE10358, p=2.31×10-06 for GSE12417 (U133plus2.0), and p=5.01×10-10 for GSE12417 (U133A)). Further, differentially expressed genes were intersected from the Hlx overexpression or inhibition studies with analogously differentially expressed genes in “HLX high” versus “HLX low” patients of the GSE14468 data set, and analyzed these genes for association with survival. Thereby, an HLX-dependent core set of 17 genes (referred to as “HLX signature”) was defined correlating with HLX expression status in patients with AML (FIG. 20, upper left panel). When patients were dichotomized into “HLX signature high” versus “HLX signature low” patients (defined by the genes of the signature, excluding HLX), it was found that “HLX signature high” patients had significantly inferior overall survival (p=0.0089 (log-rank); hazard ratio (HR)=0.66 (95% confidence interval: 0.48 to 0.90); median survival: 17.22 months for HLX signature high, not reached for HLX signature low; 5-yr survival rate: 34.5% for HLX signature high, 53.9% for HLX signature low) (FIG. 20, upper right panel). To validate in an independent cohort of patients, the HLX signature was tested in the GSE10358 data set. It was found that the signature correlated strongly (p=7.8×10-11) with “HLX high” versus “HLX low” expression status in AML patients of that cohort (FIG. 20, lower left panel). Furthermore, “HLX signature high” patients showed a strikingly inferior overall survival (p=1.89×10-05 (log-rank); hazard ratio (HR)=0.42 (95% confidence interval: 0.28 to 0.62); median survival: 18.3 months for HLX signature high, not reached for HLX signature low; 5-yr survival rate: 29.0% for HLX signature high, 67.0% for HLX signature low) (FIG. 20, lower right panel). Taken together, these data suggest that elevated HLX levels cause a specific functionally critical gene expression signature in human AML and define a disease subgroup with distinct biological properties.

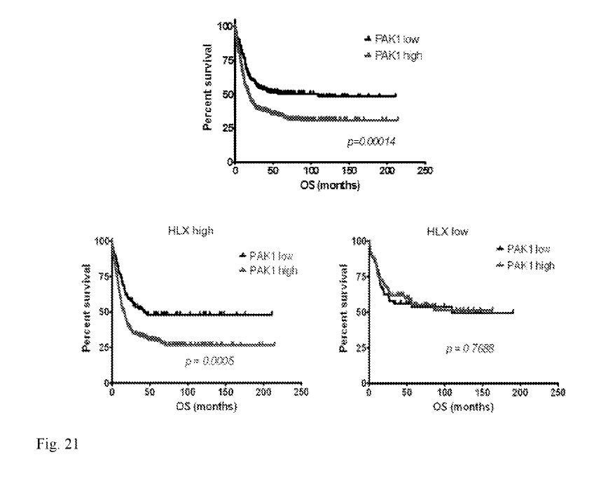

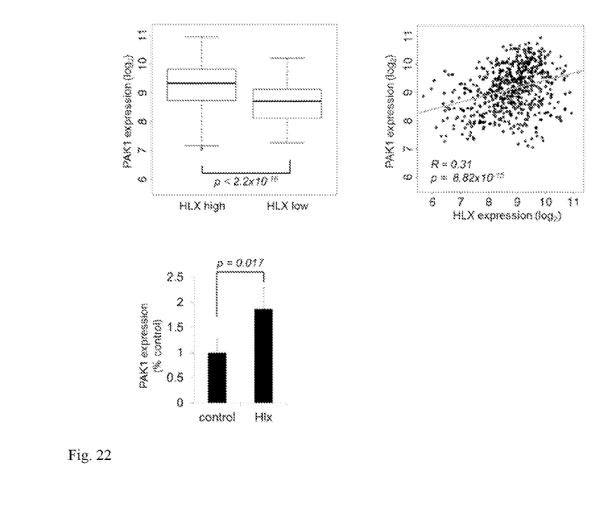

Interestingly, PAK1 was part of the HLX-induced prognostic signature in AML patients. Given the finding that PAK1 mediates the leukemia-inhibitory effects of HLX knockdown in AML cells ex vivo (FIG. 19), it was investigated whether PAK1 expression levels alone may be functionally relevant in AML patients. AML patients were dichotomized into “PAK1 high” and “PAK1 low” expressers and the clinical outcome analyzed. The “PAK1 high” patients showed significantly inferior overall survival (p=0.00014 (log-rank)) than the “PAK1 low” patients (median: 17.7 months (PAK1 high) vs. 109.1 months (PAK1 low); 5-yr survival rate: 34.0% (PAK1 high) vs. 50.5% (PAK1 low)) (FIG. 21, upper panel). Notably, high PAK1 expression was associated with inferior overall survival only in patients of the “HLX high” group (p=0.0005 (log-rank); hazard ratio (HR)=0.62 (95% confidence interval: 0.48-0.81)); median survival: 15.8 months for PAK1 high, 42.0 months for PAK1 low; 5-yr survival rate: 29.7% for PAK1 high, 48.1% for PAK1 low), but not in the “HLX low” patients (p=0.77 (log-rank); hazard ratio (HR)=1.08 (95% confidence interval: 0.65-1.78)); 5-yr survival rate: 55.0% for PAK1 high, 55.0% for PAK1 low) (FIG. 21, lower panels). In addition, PAK1 expression levels were on average 1.5-fold higher (p=2.2×10-16) in patients of the “HLX high” group compared to “HLX low” patients (FIG. 22, upper left panel). When HLX and PAK1 gene expression was analyzed in individual patients, it was also found that a significant positive correlation of HLX and PAK1 expression existed (p=8.8×10-15, R=0.31; slide 6, upper right panel). In line with this observation, experimental overexpression of Hlx in LSK cells led to a significant increase in Pak1 mRNA expression as determined by qRT-PCR (1.9-fold, p=0.017; FIG. 22 lower panel), providing further evidence that Pak1 is a functionally critical gene downstream of Hlx.

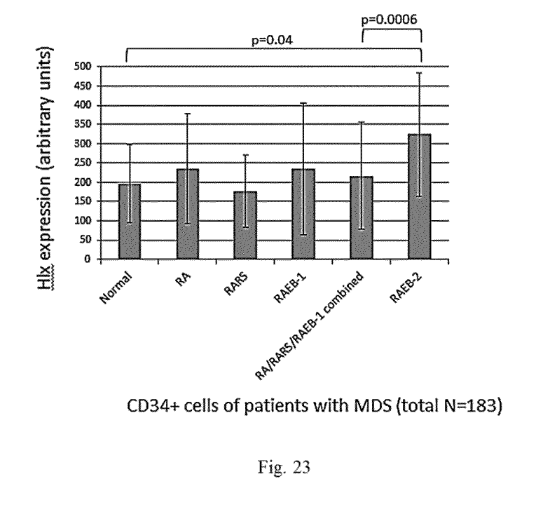

HLX was also found to be specifically elevated in patients with high-risk myelodysplastic syndromes (MDS) in a subset of patients classified as RAEB-2 (refractory anemia with excess of blasts 2) (FIG. 23). This subgroup has the most aggressive type of disease and is most likely to progress to overt AML. HLX elevation can be used to identify patients who are most likely to progress to AML and thus require treatment and can be a therapeutic target in MDS patients in general, too.

### REFERENCES

Figueroa, M. E. et al. DNA methylation signatures identify biologically distinct subtypes in acute myeloid leukemia. Cancer. Cell. 17, 13-27 (2010).
Argiropoulos, B. & Humphries, R. K. Hox genes in hematopoiesis and leukemogenesis. Oncogene 26, 6766-6776 (2007).
Alcalay, M. et al. Acute myeloid leukemia bearing cytoplasmic nucleophosmin (NPMc+AML) shows a distinct gene expression profile characterized by up-regulation of genes involved in stem-cell maintenance. Blood 106, 899-902 (2005).
Ayton, P. M. & Cleary, M. L. Transformation of myeloid progenitors by MLL oncoproteins is dependent on Hoxa7 and Hoxa9. Genes Dev. 17, 2298-2307 (2003).
Horton, S. J. et al. Continuous MLL-ENL expression is necessary to establish a “Hox Code” and maintain immortalization of hematopoietic progenitor cells. Cancer Res. 65, 9245-9252 (2005).
Bullinger, L. et al. Use of gene-expression profiling to identify prognostic subclasses in adult acute myeloid leukemia. N. Engl. J. Med. 350, 1605-1616 (2004).
Sauvageau, G. et al. Overexpression of HOXB3 in hematopoietic cells causes defective lymphoid development and progressive myeloproliferation. Immunity 6, 13-22 (1997). Thorsteinsdottir, U. et al. Overexpression of HOXA10 in murine hematopoietic cells perturbs both myeloid and lymphoid differentiation and leads to acute myeloid leukemia. Mol. Cell. Biol. 17, 495-505 (1997).
Kroon, E. et al. Hoxa9 transforms primary bone marrow cells through specific collaboration with Meis1a but not Pbx1b. EMBO J. 17, 3714-3725 (1998).
Fischbach, N. A. et al. HOXB6 overexpression in murine bone marrow immortalizes a myelomonocytic precursor in vitro and causes hematopoietic stem cell expansion and acute myeloid leukemia in vivo. Blood 105, 1456-1466 (2005).
Sauvageau, G. et al. Overexpression of HOXB4 in hematopoietic cells causes the selective expansion of more primitive populations in vitro and in vivo. Genes Dev. 9, 1753-1765 (1995).
Antonchuk, J., Sauvageau, G. & Humphries, R. K. HOXB4-induced expansion of adult hematopoietic stem cells ex vivo. Cell 109, 39-45 (2002).
Pineault, N., Abramovich, C., Ohta, H. & Humphries, R. K. Differential and common leukemogenic potentials of multiple NUP98-Hox fusion proteins alone or with Meis1. Mol. Cell. Biol. 24, 1907-1917 (2004).
Moens, C. B. & Selleri, L. Hox cofactors in vertebrate development. Dev. Biol. 291, 193-206 (2006).
Rosenbauer, F. & Tenen, D. G. Transcription factors in myeloid development: balancing differentiation with transformation. Nat. Rev. Immunol. 7, 105-117 (2007).
Rosenbauer, F. et al. Acute myeloid leukemia induced by graded reduction of a lineage-specific transcription factor, PU.1. Nat. Genet. 36, 624-630 (2004).
Steidl, U. et al. A distal single nucleotide polymorphism alters long-range regulation of the PU.1 gene in acute myeloid leukemia. J. Clin. Invest. 117, 2611-2620 (2007).
Steidl, U. et al. Essential role of Jun family transcription factors in PU.1 knockdown-induced leukemic stem cells. Nat. Genet. 38, 1269-1277 (2006).
Edgar, R., Domrachev, M. & Lash, A. E. Gene Expression Omnibus: NCBI gene expression and hybridization array data repository. Nucleic Acids Res. 30, 207-210 (2002). Ley, T. J. et al. DNMT3A mutations in acute myeloid leukemia. N. Engl. J. Med. 363, 2424-2433 (2010).
Gentles, A. J., Plevritis, S. K., Majeti, R. & Alizadeh, A. A. Association of a leukemic stem cell gene expression signature with clinical outcomes in acute myeloid leukemia. JAMA 304, 2706-2715 (2010).

What is claimed is:

1. A method of treating a preleukemia or a hematological cancer in a subject comprising administering to the subject an agent which inhibits expression of a PAK1 gene or an agent which inhibits activity of an expression product of a PAK1 gene, so as to thereby treat the preleukemia or hematological cancer.

2. The method of claim 1, wherein the agent inhibits expression of a PAK1 gene.

3. The method of claim 1, wherein the agent a small organic molecule of less than 2000 daltons, or an antibody directed against PAK1 or a fragment of said antibody.

4. The method of claim 2, wherein the agent is a nucleic acid molecule that effects RNAi and is directed to the PAK1 gene.

5. The method of claim 2, wherein the agent is an siRNA or an shRNA directed to the PAK1 gene.

6. The method of claim 1, wherein the method is for treating the preleukemia.

7. The method of claim 6, wherein the preleukemia is myelodysplastic syndrome.

8. The method of claim 1, wherein the hematological cancer is a leukemia.

9. The method of claim 8, wherein the leukemia is Acute Myeloid Leukemia.


SEQUENCE LISTING (19 sequences)

SEQ ID NO: 1 - DNA, 2308 bp, HOMO SAPIENS
aaaactttgggagtttttagagacgagtttttttttttttctattacttttccccccccctaactaacggactattattgttgttgttttaaatttagctcttagggcttagctatttgggttttcttgcggtgtccggctcccgtctccctggctcccccgcccgccctgcggccccagcgcccctcgctctcatccagcccgcgaggagtgcgggcgccgcgccgcctttaaagcgaggccagggagcgaggcggtgaccggccgagatccggccctcgcctcctccctcggtggcgctagggctcccggcctctcttcctcagtgcgggcggagaagcgaaagcggatcgtcctcggctgccgccgccttctccgggactcgcgcgcccctccccgcgcgcccacccacccagtccggctggactgcggcagccgcgcggctcaccccggcaggatgttcgcagccgggctggctcccttctacgcctccaacttcagcctctggtcggccgcttactgctcctcggccggcccaggcggctgctccttccccttggaccccgccgccgtcaaaaagccctccttctgcatcgcagacattctgcacgccggcgtgggggatctgggggcggccccggagggcctggcaggggcctcggccgccgccctcaccgcgcacttgggctcggttcacccgcacgcctctttccaagcggcggccagatccccgcttcgacccaccccagtggtggcgccctccgaagtcccggctggcttcccgcagcggctgtctccgctctcagccgcctaccaccaccatcacccgcaacaacaacagcagcagcaacagccgcagcagcaacagcctccgcctccgccccgggctggcgccctgcagcccccggcctcggggacgcgagtggttccgaacccccaccacagtggctctgccccggccccctccagcaaagacctcaaatttggaattgaccgcattttatctgcagaatttgacccaaaagtcaaagaaggcaacacgctgagagatctcacttccctgctaaccggtgggcggcccgccggggtgcacctctcaggcctgcagccctcggccggccagttcttcgcatctctagatcccattaacgaggcttctgcaatcctgagtcccttaaactcgaacccaagaaattcagttcagcatcagttccaagacacgtttccaggtccctatgctgtgctcacgaaggacaccatgccgcagacgtacaaaaggaagcgttcatggtcgcgcgctgtgttctccaacctgcagaggaaaggcctggagaaaaggtttgagattcagaagtacgtgaccaagccggaccgaaagcagctggcggcgatgctgggcctcacggacgcacaggtgaaggtgtggttccagaaccggcggatgaagtggcggcactccaaggaggcccaggcccaaaaggacaaggacaaggaggctggcgagaagccatcaggtggagccccggctgcggatggcgagcaggacgagaggagccccagccgttctgaaggcgaggctgagagcgagagcagcgactccgagtccctggacatggcccccagcgacacggagcggactgaggggagtgagcgttctctgcaccaaacaacagttattaaggccccggtcactggcgccctcattaccgccagcagtgctgggagtggtgggagcagcggcggcggcggcaatagtttcagcttcagcagcgccagcagtcttagtagcagcagcaccagtgcgggttgcgccagcagccttggcggcggcggcgcctcggagcttctccctgcaacacagcccacagccagcagcgctcccaaaagccccgagccagcccaaggcgcgcttggctgcttatagactgtactagggcggaggggatccgggccttgcgtgcagcctcccaaccatgggctgggttttgtgcttactgtatgttggcgacttggtagggcaggagacgcagcgtggagcctacctcccgacattcacgcttcgccccacgctgctccgactggctgcagcggacactgcccaaagcagaggggagtctcagtgtcctgctagccagccgaacacttctctccggaagcaggctggttcgactgtgaggtgtttgactaaactgtttctctgactcgccccagaggtcgtggctcaaaggcacttaggacgccttaaatttgtaaataaaatgtttactacggtttgtaaaaaaaaaaaaaaaaaaaaaaaaaaaaaaaa

SEQ ID NO: 2 - DNA, 23 bp, ARTIFICIAL SEQUENCE
  PRIMER FOR MOUSE HLX
ttcagcatcaattccaagacaca

SEQ ID NO: 3 - DNA, 22 bp, ARTIFICIAL SEQUENCE
  PRIMER FOR MOUSE HLX
acctcttctccaggccttttct

SEQ ID NO: 4 - DNA, 22 bp, ARTIFICIAL SEQUENCE
  PRIMER FOR MOUSE BTG
tcatctccaagttcctccgcac

SEQ ID NO: 5 - DNA, 22 bp, ARTIFICIAL SEQUENCE
  PRIMER FOR MOUSE BTG
caacggtaacctgatcccttgc

SEQ ID NO: 6 - DNA, 22 bp, ARTIFICIAL SEQUENCE
  PRIMER FOR MOUSE FoxOR4
tctacgaatggatggtccgcac

SEQ ID NO: 7 - DNA, 22 bp, ARTIFICIAL SEQUENCE
  PRIMER FOR MOUSE FoxOR4
cttgctgtgcaaggacaggttg

SEQ ID NO: 8 - DNA, 22 bp, ARTIFICIAL SEQUENCE
  PRIMER FOR MOUSE Gadd45a
cctggaggaagtgctcagcaag

SEQ ID NO: 9 - DNA, 21 bp, ARTIFICIAL SEQUENCE
  PRIMER FOR MOUSE Gadd45a
gtcgtcttcgtcagcagccag

SEQ ID NO: 10 - DNA, 22 bp, ARTIFICIAL SEQUENCE
  PRIMER TO MOUSE Hdac7
cgcctcaaactggataacggga

SEQ ID NO: 11 - DNA, 22 bp, ARTIFICIAL SEQUENCE
  PRIMER TO MOUSE Hdac7
gcattggaggaatgcagctcgt

SEQ ID NO: 12 - DNA, 22 bp, ARTIFICIAL SEQUENCE
  PRIMER TO MOUSE Pak1
attgctccacgcccagaacaca

SEQ ID NO: 13 - DNA, 22 bp, ARTIFICIAL SEQUENCE
  PRIMER TO MOUSE Pak1
aagcatctggcggagtggtgtt

SEQ ID NO: 14 - DNA, 21 bp, ARTIFICIAL SEQUENCE
  PRIMER TO MOUSE Satb1
cgccattgaatatgattgcaa

SEQ ID NO: 15 - DNA, 21 bp, ARTIFICIAL SEQUENCE
  PRIMER TO MOUSE Satb1
tccaacctggattagcccttt

SEQ ID NO: 16 - DNA, 18 bp, ARTIFICIAL SEQUENCE
  PRIMER DIRECTED TO MOUSE Gadph
ccagcctcgtccgtagac

SEQ ID NO: 17 - DNA, 19 bp, ARTIFICIAL SEQUENCE
  PRIMER DIRECTED TO MOUSE Gadph
gccttgactgtgccgttga

SEQ ID NO: 18 - DNA, 20 bp, ARTIFICIAL SEQUENCE
  PRIMER DIRECTED TO Trp63
tgagccgtgagttcaatgag

SEQ ID NO: 19 - DNA, 20 bp, ARTIFICIAL SEQUENCE
  PRIMER DIRECTED TO Trp 63
acctgtggtggctcataagg